(19) United States
(12) Patent Application Publication
(10) Pub. No.: US 2026/0232283 A1
(43) Pub. Date: Aug. 13, 2026

(54) SYSTEMS, METHODS, AND DEVICES FOR SIMULTANEOUS PET-SPECT IMAGING

(71) Applicant: Washington University, St. Louis, MO (US)

(72) Inventors: Yuan-Chuan TAI, St. Louis, MO (US); Yunlai CHEN, St. Louis, MO (US); Samarth AGGARWAL, St. Louis, MO (US); Ling CAI, St. Louis, MO (US)

(21) Appl. No.: 19/532,067

(22) Filed: Feb. 6, 2026

Related U.S. Application Data
(60) Provisional application No. 63/755,506, filed on Feb. 7, 2025.
(60) Provisional application No. 63/976,420, filed on Feb. 5, 2026.

(51) Int. Cl. A61B 6/03; G01T 1/202

(52) U.S. Cl. CPC A61B 6/037; G01T 1/202

(57) ABSTRACT

A dual modality system for PET and SPECT includes a first detector module to detect gamma photons for SPECT imaging of a subject from a first position and to generate first output data in response to the detections, a second detector module to detect gamma photons from a second position opposite the first position and to generate second output data in response to the detection, and a computer device in communication with the first detector module and the second detector module. The computer device includes a processor and a memory. The processor is programmed to receive the first output data, receive the second output data, perform SPECT imaging of the subject using the received first output data, and perform PET imaging of the subject using coincidence data from the received first output data and the received second output data.

### CROSS REFERENCE TO RELATED APPLICATIONS

This application claims priority to U.S. Provisional Patent Application No. 63/755,506, filed Feb. 7, 2025, and U.S. Provisional Patent Application No. 63/976,420, filed Feb. 5, 2026, each of which is hereby incorporated by reference herein in its entirety.

### BACKGROUND

The field relates generally to imaging devices, systems, and methods. Some aspects of this disclosure relate generally to devices, systems and methods of gamma-ray imaging. Some aspects of the is disclosure relate to devices, systems, and methods for performance of Single-Photon Emission Computed Tomography (SPECT) and Positron Emission Tomography (PET) imaging. At least: some embodiments are operable to perform simultaneous PET and SPECT imaging.

Over 50% of cancer patients receive radiation treatment in some form in the course of disease management. This is a staggering number given the 1.7 million new cases a year in the U.S. Therapies in the form of X-ray or proton therapy, or implanted as brachytherapy, can eradicate or provide long-term control for primary disease in many common cancers. In the metastatic setting however, highly effective conventional radiotherapy is mostly limited to palliative applications. An emerging class of internal radiotherapies using radiolabeled molecules that are systemically administered and localize to sites of disease have generated intense academic, industrial and clinical excitement.

Alpha-emitting Radiopharmaceutical Therapy (αRPT) is an emerging internal radiotherapy that sparks significant interest due to the advantages of alpha particles' densely ionizing track and a short path length of only several cells. Conjugated with molecularly targeting agents, this therapy with high linear energy transfer (LET) leads to severe DNA double strand breaks to tumor cells, while sparing adjacent normal tissues. Other benefits of αRPT such as imperviousness to resistance and independence of oxygenation make it superior to standard therapy.

Optimization of αRPT requires quantitative measurement of the α-emitting radiopharmaceutical distribution to estimate the absorbed dose to tumors and in vital organs. The administered dose of αRPT activities are at least two to three orders of magnitude lower than those used with diagnostic radiopharmaceutical imaging procedures. This creates a challenge for imaging αRPT using the current nuclear medicine imaging technique, specifically SPECT, because its sensitivity is inherently hindered by the use of heavy metal (e.g., lead) collimators, which blocks >99% of incoming photons.

Extensive efforts have been made to enhance SPECT system sensitivity. Innovations include the AdaptiSPECT-C scanner with adjustable pinhole sizes via a shutter mechanism, and the exploration of non-traditional apertures like micro-slit and micro-ring collimators. Coded aperture (CA) designs, which offer a favorable balance between resolution and sensitivity, have shown promising results in several studies. Hybrid approaches combining CA and Compton imaging have also been developed to image and quantify Ac-225 and its daughters in mice. In addition to collimator advancements, SPECT systems based on semiconductor detectors such as Cadmium Zinc Telluride (CZT) are gaining attention for their superior energy resolution (<5% at 140 keV). CZT detectors with high intrinsic spatial resolution can be combined with multi-pinhole collimators or Compton imaging mode to design SPECT cameras with high system sensitivity.

However, measurements of radiation dosimetry from αRPT alone are insufficient for treatment optimization. Poly ADP-ribose polymerase 1 (PARP1) is an enzyme that is rapidly recruited and activated by double-strand DNA breaks and it plays a critical role in DNA repairs. Tumor cells that upregulate PARP1 following αRPT may develop resistance. Therefore, assessment of the PARP1 status in target cancer cells is emerging as a means to guide and personalize treatments, for example, through inclusion of a PARP1 inhibitor to enhance αRPT. To enable non-invasive monitoring of PARP1 expression, [¹⁸F]PARPZ was developed as a PET imaging ligand for real-time visualization and quantification of PARP1 activity. PARP-PET imaging provides valuable insights into the genotoxic effects of αRPT, allowing clinicians to predict treatment response, optimize combination therapy timing, and serve as a quantitative in vivo biodosimeter.

To investigate the interplay between αRPT and DNA repair activities, a system for in vivo ultrasensitive SPECT imaging of radiation dose from αRPT and PET imaging of PARP1-targeting agent simultaneously would be beneficial. Simultaneous SPECT/PET imaging has been investigated in where PET systems are modified by inserting multi-pinhole collimators, or the incorporation of multiple collimators optimized for low- and high-energy gamma-rays into SPECT systems for SPECT and PET radiopharmaceuticals. These approaches rely on heavy metal collimator limiting their potential for αRPT imaging which requires two to three orders of magnitude increase in system sensitivity. They also have low system sensitivity for PET imaging when compared to traditional PET scanners that utilize coincidence detection for electronic collimation instead of heavy metal collimators.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

One aspect of the disclosure is a dual modality imaging system for positron emission tomography (PET) and single-photon emission computed tomography (SPECT). The system comprises a first detector module configured to detect gamma photons for SPECT imaging of a subject from a first position proximate the subject without using a heavy-metal collimator and to generate first output data in response to gamma photons detected by the first detector module, a second detector module configured to detect gamma photons from a second position proximate the subject opposite the first position and to generate second output data in response to gamma photons detected by the second detector module, and a computer device in communication with the first detector module and the second detector module. The computer device comprises at least one processor in communication with at least one memory device. The at least one processor is programmed to receive the first output data from the first detector module, receive the second output data from the second detector module, perform SPECT imaging of the subject using the received first output data, and perform PET imaging of the subject using coincidence data from the received first output data and the received second output data.

According to another aspect of the disclosure, a method for positron emission tomography (PET) and single-photon emission computed tomography (SPECT) imaging includes positioning a first detector module to receive first gamma photons from a subject, the first detector module comprising a coded sensor gamma imager module including a spatial-encoding sensor element comprising scintillator material arranged to have a nonuniform structure in at least one dimension and a scintillation crystal layer positioned below the spatial-encoding sensor element. A second detector module is positioned to receive second gamma photons from the subject and the received first gamma photons are collimated using the spatial-encoding sensor element. The first gamma photons are detected by the spatial-encoding sensor element or by the scintillation crystal layer after the gamma photons pass through the spatial-encoding sensor element and first data on the first gamma photons that are detected by the first detector module is output to a computing device. Second gamma photons are detected with the second detector module and second data on the second gamma photons that are detected by the second detector module is outputting to the computing device and synchronized with the first data. The method includes one or both of: reconstructing, by the computing device, a SPECT image based at least in part on the first data, and reconstructing, by the computing device, a PET image based at least in part on the first data and the second data.

Another aspect of this disclosure is a method of calibrating a first gamma photon detector module with respect to a second gamma photon detector module. The method includes positioning the first gamma photon detector module opposite the second gamma photon detector module and positioning a positron emitting source between the first gamma photon detector module and the second gamma photon detector module at a known first distance from the first gamma photon detector module and a known second distance from the second gamma photon detector module. The first gamma photon detector module includes at least a light photon sensor coupled to at least an end of the first gamma photon detector module. Coincidence events of annihilation gamma photons from the positron source interacting with the first gamma photon detector module and the second gamma photon detector module are recorded and timing factors based on the recorded coincidence events are determined. The timing factors include one or more timing offsets for timing correction of the first gamma photon detector module, and one or more weighting factors for deriving trigger times of the coincidence events.

Various refinements exist of the features noted in relation to the above-mentioned aspects. Further features may also be incorporated in the above-mentioned aspects. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following figures illustrate various aspects of the disclosure.

FIG. 1 is a dual modality system for performing PET and SPECT imaging.

FIG. 2 is a simplified block diagram of a computing device that may be used in the system of FIG. 1.

FIG. 3A is a top view of an example broadband ultrasensitive gamma imager (BUGI) module for use in the system of FIG. 1.

FIG. 3B is a cross-section view along the line A-A of the BUGI module shown in FIG. 3A.

FIG. 4A is an isometric view of another example BUGI module.

FIG. 4B is a simplified cross-sectional view of the BUGI module shown in FIG. 4A taken along the X axis as viewed from the Y direction in FIG. 4A.

FIG. 4C is the simplified cross-sectional view of FIG. 4B with the paths of example gamma photons shown as dashed arrows and indicating detection events.

FIG. 5 is a simplified view of six scintillation crystals of a portion of a coded sensor (CS) element for the gamma camera module of FIGS. 4A-4C.

FIG. 6 is a gamma ray distribution on a sidewall of the BUGI module of FIGS. 4A-4C.

FIG. 7 shows a gamma-ray from the source E0 undergoing Compton scattering at E1 followed by a photoelectric interaction at E2 in the BUGI module of FIGS. 4A-4C.

FIG. 8A is the five types of crystal columns used to construct an example BUGI module.

FIG. 8B is a top view of an example BUGI module constructed using the five types of crystal columns shown in FIG. 8A.

FIG. 9A is a prototype BUGI module and associated readout circuitry.

FIG. 9B is the prototype BUGI module of FIG. 9A mounted in a housing.

FIG. 10 is an analytical model of the system response function of a prototype BUGI module, where a representative scintillation crystal at rd is irradiated by a point source located at r0.

FIG. 11 is a map of histograms of energy ratio measured by the 64 crystal columns when an example BUGI module of FIG. 9B was irradiated by a Na-22 source.

FIG. 12 is an example embodiment of the system shown in FIG. 1 in which the first detector module is a BUGI detector module and the second detector module is a conventional TOF-PET detector crystal array.

FIG. 13 an example embodiment of the system shown in FIG. 1 in which the first detector module 135 and the second detector module 137 are both BUGI detector modules.

FIG. 14 is a setup for detector calibration.

FIG. 15 is an energy ratio plot of one type of crystal columns.

FIG. 16 is an energy ratio plot of another type of crystal columns.

FIG. 17 is a flow chart of the depth-dependent timing alignment framework.

FIG. 18 is a test setup with a BUGI detector module and a TOF-PET detector module positioned to image a hot-rod phantom.

FIG. 19 is an image of a cuboid hot-rod phantom filled with Cu-64 solution for SPECT and PET imaging studies.

FIG. 20 is an image of a cylindrical hot-rod phantom used for simultaneous PET and SPECT imaging studies. The outer 3 cylindrical rods are filled with Tc-99m solution while the inner 3 cylindrical rods are filled with Cu-64 solution.

FIG. 21 includes representative count distributions from three parts of a BUGI detector module corresponding to a Tc-99m point source location at (0,0,150) mm.

FIG. 22 includes representative count distributions from three parts of a BUGI detector module corresponding to a Tc-99m point source location at (50,50,150) mm.

FIG. 23 is the sensitivity images of the three subsets of detectors in a BUGI module: bottom camera, spatial-encoding sensors, and side wall detectors. The surface plots are calculated using the system model in FIG. 10 while the Monte Carlo simulation results are presented as symbols overlaid on the surface plots.

FIG. 24 is energy ratio histograms from different radioactive sources: Na-22, Tc-99m, Lu-177, and Pb-212 as detected by the five different types of crystal columns shown in FIG. 8A.

FIG. 25 includes graphs of representative energy spectra of the 2-layer GAGG crystals in a detector column of type B shown in FIG. 8A.

FIG. 26 includes graphs related to timing calibration when studying the system shown in FIG. 18.

FIG. 27 is 2D distributions of the full-width-at-half-maximum (FWHM) of the timing spectrum for each crystal element in a BUGI detection module at eight DOI layers.

FIG. 28 is a system geometry of a BUGI SPECT system for imaging the phantom in FIG. 19 using the test setup in FIG. 18.

FIG. 29 shows the reconstructed SPECT 3D image of the phantom in FIG. 19 using the test setup in FIG. 18 and the BUGI SPECT system geometry of FIG. 28.

FIG. 30 is SPECT images of the same phantom as in FIG. 29 acquired using a known clinical SPECT system.

FIG. 31A is a sensitivity image of the test setup in FIG. 18 for PET imaging using the BUGI detector module prototype in coincidence with a TOF PET Detector for imaging at a single angle.

FIG. 31B is a sensitivity image of the test setup in FIG. 18 for PET imaging using the BUGI detector module prototype in coincidence with a TOF PET Detector for imaging at 7 angles in 30° step increments.

FIG. 32 is the reconstructed PET images of the same phantom as in FIG. 29 in transverse, coronal and sagittal views.

FIG. 33 is simultaneous dual-isotope PET-SPECT imaging results of the phantom in FIG. 20 using the test setup in FIG. 18.

Corresponding reference characters indicate corresponding parts throughout the drawings.

### DETAILED DESCRIPTION

This disclosure relates generally to imaging devices, systems, and methods. Some aspects of this disclosure relate generally to devices, systems and methods of gamma-ray imaging. Some aspects of this disclosure relate to devices, and methods for Single-Photon Emission Computed Tomography (SPECT) and Positron Emission Tomography (PET).

To achieve simultaneous ultrasensitive SPECT and PET imaging, embodiments of this disclosure may utilize one or more a Broadband Ultrasensitive Gamma Imager (BUGI) module. The BUGI modules may sometimes be referred to as coded sensor gamma imager (CSGI) modules. Example BUGI modules may incorporate combinations of the geometry and functionalities of multi-pinhole SPECT, coded aperture cameras, self-collimating imaging, and Compton camera into a single device. The proposed BUGI designs offer higher system sensitivity than both the conventional collimator and coded aperture-based systems because it encodes and detects (as opposed to rejecting) gamma-rays (γ-rays) when acquiring SPECT images. When built with fast scintillator material, the BUGI detector modules are also operable as time-of-flight PET (TOF-PET) detectors and thus further enable PET imaging at the same time and with the same device as SPECT imaging.

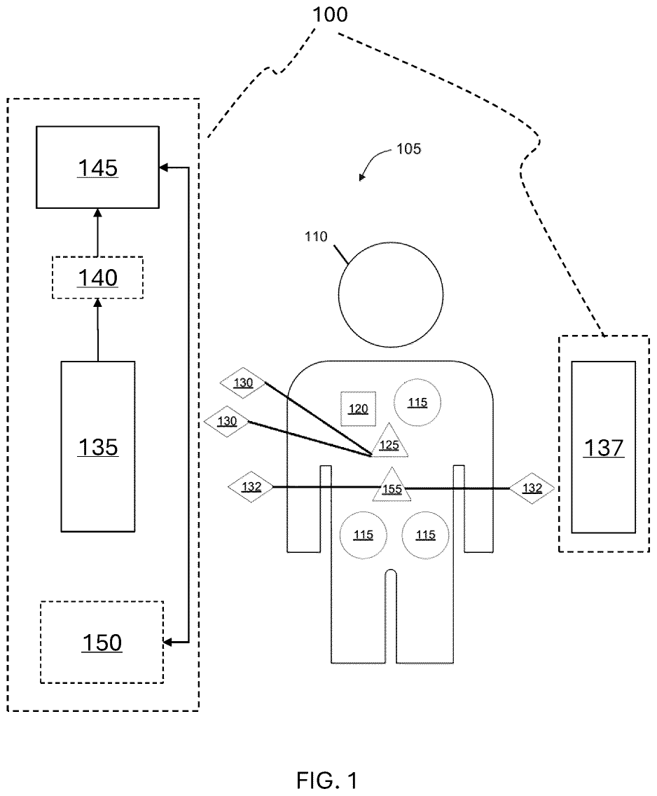

FIG. 1 is a dual modality system 100 for performing PET-SPECT imaging. In the example embodiment, the system is being used in a radiopharmaceutical therapy environment 105, but the system may be used in any environment suitable for and for any purposes benefiting from PET and or SPECT imaging. The system 100 may sometimes be referred to as a Simultaneous and Ultrasensitive Photon and Positron Imaging (SUPRIM) system.

In the exemplary embodiment, a patient 110 has a plurality of organs 115, where one or more of the organs 115 are radio sensitive. The patient 110 also has one or more tumors 120. The one or more tumors 120 are being treated with an alpha particle-emitting radiopharmaceutical therapy (α-RPT), which is shown in FIG. 1 dispersed through the patient 110 after administration as α-RPT 125. The α-RPT 125 emits alpha (α) particles, which are absorbed by the surrounding tissue, and gamma (γ)-ray photons 130, which exit the patient's body. A first detector module 135 detects the γ-ray photons 130. The first detector module 135 is configured to detect gamma photons for SPECT imaging of the patient subject from a first position proximate the subject without using a heavy-metal collimator and to generate first output data in response to gamma photons detected by the first detector module. In the example embodiment, the first detector module 135 is a BUGI module. The first detector module 135 is coupled to a computer system 145 for receiving data from the first detector module 135 and performing the calculations described herein to detect the location of the α-RPT 125 based on the detected γ-ray photons 130 and to reconstruct an image based on the data received from the BUGI module. Although a single first detector module 135 is shown, the system 100 may include more than one first detector module 135. In some embodiments, the first detector module 135 is coupled to the computer system 145 via one or more readout circuit 140. In other embodiments, suitable readout circuitry is incorporated in the first detector module 135 and separate readout circuitry 140 may not be needed.

While a conventional pinhole collimated gamma camera includes a collimator constructed with heavy metal such that it blocks >99% of incoming photons while allowing for only those aligned with specific direction to travel through the apertures, BUGI detector modules substitute heavy metal with scintillator material thereby integrating the collimator as a part of the sensor and accepting all photons that interact with the BUGI detector module. To maximize the overall information content of the detected events, the scintillation crystals are arranged strategically in 3D to encode and extract directional, spatial and energy information. An example BUGI detector module builds on a geometry that can be divided into 3 sub-groups designed to illustrate different functionalities: (1) A bottom layer of scintillation crystal is laid out underneath a spatial-encoding sensor element, which resembles a geometry similar to a pinhole camera or a coded-aperture camera. (2) The central region is a spatial-encoding sensor element comprising scintillator material arranged to form a nonuniform structure that serves dual purposes of directional selectivity and small active sensors, named as Coded Sensors (CS). Crystals located at the lower layers of CS will detect gamma-rays that contain more directional information to decode the location of the source than those above, while crystal elements at top layers will provide both spatial encoding and increased sensitivity. (3) Crystal elements on the 4 side walls (in, for example, four-sided embodiments) function as a coded aperture camera. Incoming gamma rays from an oblique angle will project a checkerboard pattern on these 4 side walls. Effectively, a single BUGI module incorporates the functionality and geometrical design of a conventional pinhole gamma camera, a self-collimation camera and four coded-aperture cameras, resulting in substantially more counts and information content for ultra-high sensitivity SPECT imaging.

To enable simultaneous imaging of gamma-emitting and positron-emitting radionuclides, the BUGI detector module can be built with fast scintillator material to support TOF-PET imaging capability. The choice of scintillation material often requires balance between detector efficiency for system sensitivity and fast timing response for TOF-PET imaging. To acquire SPECT images at extreme low counts, scintillators that are free of self-radiation may be beneficial. Thus, some preferred embodiments use gadolinium aluminum gallium garnet (GAGG) crystals, which offer high light yield, high density, short decay time, and absence of intrinsic radioactivity making it well-suited for use in the SUPRIM systems. The BUGI modules and their construction will be described in more detail below.

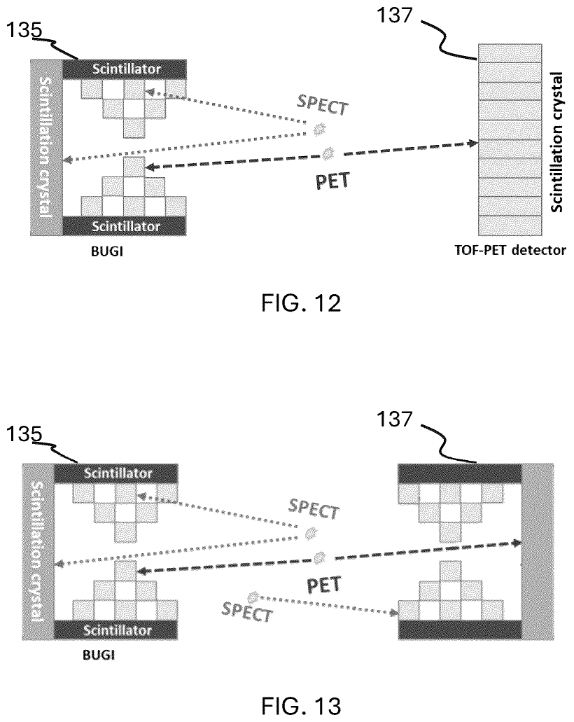

A positron emitting source 155 emits positrons, each of the emitted positrons that collides with and annihilates an electron produces a pair of 511 keV gamma photons 132. In some embodiments, the positron emitting source 155 is a positron radionuclide, such as ¹⁸F or ¹¹C. In other embodiments, they may be any other suitable radiation source usable for PET imaging. The paired gamma photons 132 are emitted approximately one-hundred and eighty degrees apart. The paired gamma photons intersect and are detected by the first module 135 and a second detector module 137. The second detector module 137 is configured to detect gamma photons from a second position proximate the subject opposite the first position and to generate second output data in response to gamma photons detected by the second detector module. In some embodiments, the second detector module 137 is a standard TOF-PET detector, such as a TOF-PET detector scintillation crystal array. In other embodiments, the second detector module 137 is the same type of detector as the first detector module 135, such as a BUGI module. FIG. 12 illustrates an embodiment in which the first detector module 135 is a BUGI detector module and the second detector module 137 is a conventional TOF-PET detector crystal array. FIG. 13 illustrates an embodiment in which the first detector module 135 and the second detector module 137 are both BUGI detector modules.

It should be understood that when the first and second detector modules 135, 137 are both BUGI modules, each of the first and second detector modules can be individually used for SPECT imaging and the two detector modules can be cooperatively used for PET imaging. It should further be understood that although only two detector modules are shown, that is the minimum number of detector module needed and not the only number or a maximum number of module. Rather, the system 100 may include any number of detector modules greater than two as long as it includes at least one BUGI module to allow simultaneous PET and SPECT imaging.

In some embodiments, the system 100 includes, or is in communication with a second imaging modality such as a computed tomography (CT) or a magnetic resonance imaging (MRI) or an ultrasound imaging (UI) device 150. The CT (or MRI or UI) device 150 may be a CT (or MRI or UI) machine or CT (or MRI or UI) scanning machine. The CT (or MRI or UI) device 150 provides scans of the patient, or item being imaged, to allow the system 100 to determine where on the patient's body the photons originated. In some embodiments, the computing device 145 controls the CT (or MRI or UI) device 150 to produce the scans of the patient.

The computing device 145 is in communication with the first detector module 135 and the second detector module 137. The computing device is programmed to receive the first output data from the first detector module, receive the second output data from the second detector module, perform SPECT imaging of the subject using the received first output data, and perform PET imaging of the subject using coincidence data from the received first output data and the received second output data.

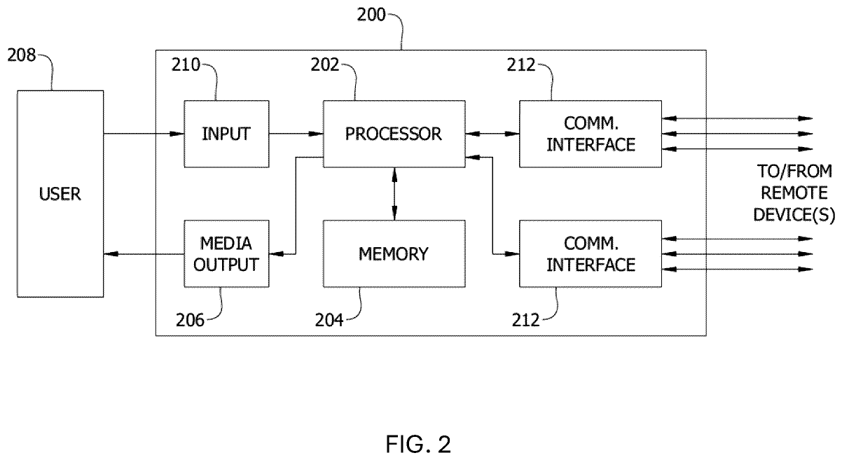

Turning to FIG. 2, an example configuration of a computing device 200 that may be used as the computing device 145 and/or any other computers, computing device, controllers, or the like described herein is shown. The computing device 200 includes a processor 202, a memory 204, a media output component 206, an input device 210, and communications interfaces 212. Other embodiments include different components, additional components, and/or do not include all components shown in FIG. 2.

The processor 202 is configured for executing instructions. In some embodiments, executable instructions are stored in the memory 204. The processor 202 may include one or more processing units (e.g., in a multi-core configuration). As used herein, the term “processor” refers not only to integrated circuits, but also to a controller, a microcontroller, a microcomputer, a programmable logic controller (PLC), an application-specific integrated circuit, a graphic processing unit, and other programmable circuits. The memory 204 may generally be or include memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable non-transitory memory elements and is generally any device allowing information such as executable instructions and/or other data to be stored and retrieved. Such memory 204 may generally be configured to store suitable computer-readable instructions that, when implemented by the processor 202, configure, cause, or program the computing device 200 to perform various functions described herein.

The media output component 206 is configured for presenting information to user 208. The media output component 206 is any component capable of conveying information to the user 208. In some embodiments, the media output component 206 includes an output adapter such as a video adapter and/or an audio adapter. The output adapter is operatively connected to the processor 202 and operatively connectable to an output device such as a display device (e.g., a liquid crystal display (LCD), organic light emitting diode (OLED) display, cathode ray tube (CRT), “electronic ink” display, one or more light emitting diodes (LEDs)) or an audio output device (e.g., a speaker or headphones).

The computing device 200 includes, or is connected to, the input device 210 for receiving input from the user 208. The input device is any device that permits the computing device 200 to receive analog and/or digital commands, instructions, or other inputs from the user 208, including visual, audio, touch, button presses, stylus taps, etc. The input device 210 may include, for example, a variable resistor, an input dial, a keyboard/keypad, a pointing device, a mouse, a stylus, a touch sensitive panel (e.g., a touch pad or a touch screen), a gyroscope, an accelerometer, a position detector, or an audio input device. A single component such as a touch screen may function as both an output device of the media output component 206 and the input device 210.

The communication interfaces 212 enable the computing device 200 to communicate with remote devices and systems, such as allowing communication between the computing device 145 and the first detector module 135, the CT (or MRI or UI) device 150, remote computing devices or servers (not shown), and the like. The communication interfaces 212 may be wired or wireless communications interfaces that permit the computing device to communicate with the remote devices and systems directly or via a network. Wireless communication interfaces 212 may include a radio frequency (RF) transceiver, a Bluetooth® adapter, a Wi-Fi transceiver, a ZigBee® transceiver, a near field communication (NFC) transceiver, an infrared (IR) transceiver, and/or any other device and communication protocol for wireless communication. (Bluetooth is a registered trademark of Bluetooth Special Interest Group of Kirkland, Washington; ZigBee is a registered trademark of the ZigBee Alliance of San Ramon, California.) Wired communication interfaces 212 may use any suitable wired communication protocol for direct communication including, without limitation, USB, I2C, RS232, SPI, analog, and proprietary I/O protocols. In some embodiments, the wired communication interfaces 212 include a wired network adapter allowing the computing device to be coupled to a network, such as the Internet, a local area network (LAN), a wide area network (WAN), a mesh network, and/or any other network to communicate with remote devices and systems via the network. Although two communication devices 212 are shown, the computing device 200 may include more or fewer computing devices.

It should be understood that in some embodiments the computing device 200 does not include or use an input 210 or a media output 206 and a user 208 may not directly interact with the computing device. Rather, the user 208 (or another computing device) may only interact remotely with computing device 200 through the communication interface 212.

Moreover, in some embodiments the computing device 200, or parts thereof, may not be a physical computing device local to the user 208, but instead is cloud based. Thus, for example, the computing device 145 may be a cloud-based computing device or may be a physical computing device 200 using cloud-based storage for all or part of its memory, using cloud-based processing instead of local processing for some or all of its processing, or the like.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. As used herein, the term “cloud computing” and related terms, e.g., “cloud computing devices” refers generally to a computer architecture allowing for the use of multiple heterogeneous computing devices for data storage, retrieval, and processing. The heterogeneous computing devices may use a common network or a plurality of networks so that some computing devices are in networked communication with one another over a common network but not all computing devices. In other words, a plurality of networks may be used to facilitate the communication between and coordination of all computing devices.

The computing device 200 may be or include any suitable stationary or portable computing device, computer, desktop computer, laptop computer, tablet computer, mobile device, single-board computer, microcontroller, system-on-module, programmable logic board, or the like.

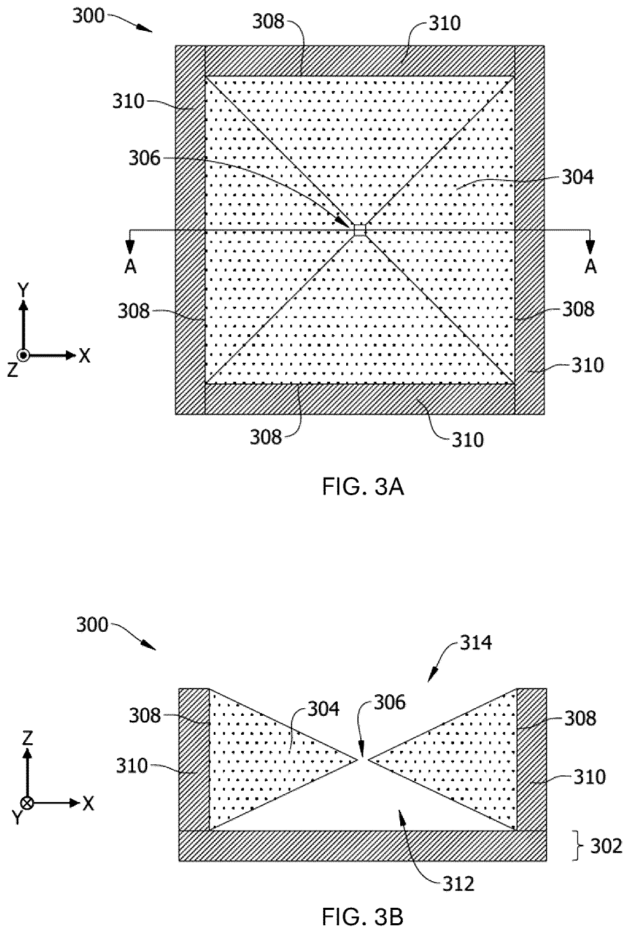

FIGS. 3A and 3B are a simplified diagrams of an example BUGI module 300 that may be used as the first detector module 135. FIG. 3A is a top view of the BUGI module and FIG. 3B is a cross-section view of the BUGI module along the line A-A in FIG. 3A. The example BUGI module is not drawn to scale and may not include all elements/components (i.e., it is “simplified”). The example BUGI module is cuboid in shape, but other embodiments may have any other suitable shape. The BUGI module 300 is specially designed for higher sensitivity than known gamma cameras used in traditional SPECT imaging generally, and for use in CSGI-SPECT imaging specifically. The BUGI module 300 does not use a heavy metal collimator as known gamma cameras do. As will be described in more detail below, rather than rejecting photons with a heavy metal collimator, the BUGI module 300 uses coded sensor element that accepts substantially all photons that interact with it and provides spatial and directional selectivity.

The BUGI module 300 includes a scintillation crystal layer 302 for detecting photons, a coded sensor element 304, and sidewalls 310. In the example embodiment, the scintillation crystal layer is composed of gadolinium aluminum gallium garnet (GAGG) scintillation crystals. Some embodiments use solid-state detectors such as CZT instead of GAGG scintillation crystals. Other embodiments may use any other suitable scintillation crystals or detector material such as gadolinium oxyorthosilicate (GSO), bismuth germanate (BGO), cadimium telluride (CdTe), or any materials that can interact with gamma rays and produce detectable signals.

The coded sensor element 304 is a spatial-encoding element positioned above (as viewed in FIG. 3B) the scintillation crystal layer. In this example embodiment, the coded sensor element also includes scintillator material arranged to form a pinhole lens having an interior pinhole aperture 306 above the scintillation crystal layer and outer edges 308. Other embodiments may use any other collimation configuration, such as parallel hole or complex coded apertures. Because the example BUGI module is cuboid in shape, it includes four outer edges. In other embodiments, the gamma camera module may have any other suitable shape with any other number of outer edges, including only one outer edge (in embodiments having a cylindrical, spherical, or hemispherical shape, for example). In additional to the scintillator material, the coded sensor element also includes a non-scintillator material. The non-scintillator material is a non-heavy metal material. In the example embodiment, the non-scintillator, non-heavy metal material is an acrylic material. Other embodiments may use other suitable material such as polycarbonate or other transparent materials that allow light photons generated in the scintillation crystals to be transmitted to the outer surfaces of the BUGI module and be detected by light sensors.

In the example embodiment, the scintillator material and the acrylic in the coded sensor element 304 are cuboid crystals and are arranged in an alternating pattern in both the X, Y, and Z directions. Thus, when viewed from above as in FIG. 3A and as viewed in cross section as in FIG. 3B, the alternating scintillator material and the acrylic in the coded sensor element would form a checkerboard pattern. In the example embodiment, the assembled scintillator material and acrylic in the coded sensor element generally have similar dimensions when viewed from the directions shown in FIGS. 3A and 3B. The spaces 312 and 314 are illustrated unfilled (or filled with the atmosphere that surrounds the BUGI module 300) in the example embodiment. In other embodiments, the spaces 312 and 314 are filled with a non-scintillator, non-heavy metal material. In such embodiments, the pinhole aperture 306 is typically also filled with the non-scintillator, non-heavy metal material. The non-scintillator, non-heavy metal material embodiments that include it in spaces 312 and 314 is any non-scintillator, non-heavy metal material that gamma photons may readily pass through with low probability of interaction. In some embodiments, the spaces 312 and 314 are filled with the same non-scintillator, non-heavy metal material that is used in the coded sensor element 304 (e.g., an acrylic). Other embodiments use a material that is different than the non-scintillator, non-heavy metal material that is used in the coded sensor element 304. In some embodiments in which signals are readout from the top and bottom, it is preferable to fill both of the spaces 312 and 314 with the same non-scintillator, non-heavy metal material (e.g., the same as is used in the coded sensor element 304). Embodiments that readout signals from only the bottom may leave the space 314 empty (i.e., filled with air or ambient atmosphere) and fill the space 312 with a non-scintillator, non-heavy metal material (e.g., the same as is used in the coded sensor element 304).

Sidewalls 310 are disposed above the scintillation crystal layer 302 and adjacent the outer edges 308 of the coded sensor element 304. Thus, the example BUGI module 300 includes four sidewalls. Other embodiments may include more or fewer sidewalls depending on the shape of the BUGI module and the number of outer edges that the coded sensor element has. Typically, it is desired that each outer edge of the coded sensor element has a sidewall disposed adjacent to it. However, some embodiments may include fewer sidewalls than coded sensor element outer edges. The sidewalls are made of scintillator material. Each sidewall works in conjunction with the coded sensor element (and specifically with the alternately arranged scintillator material and acrylic in the coded sensor element) to function as a coded aperture camera.

Thus, the BUGI module 300 functions as a pinhole camera and multiple (depending on how many sidewalls 310 are present) coded aperture cameras to detect gamma photons that reach the BUGI module. The BUGI module does this without rejecting (as a standard heavy metal collimator would) most gamma photons that reach it and provides significantly increased sensitivity and additional information as compared to at least some known gamma cameras.

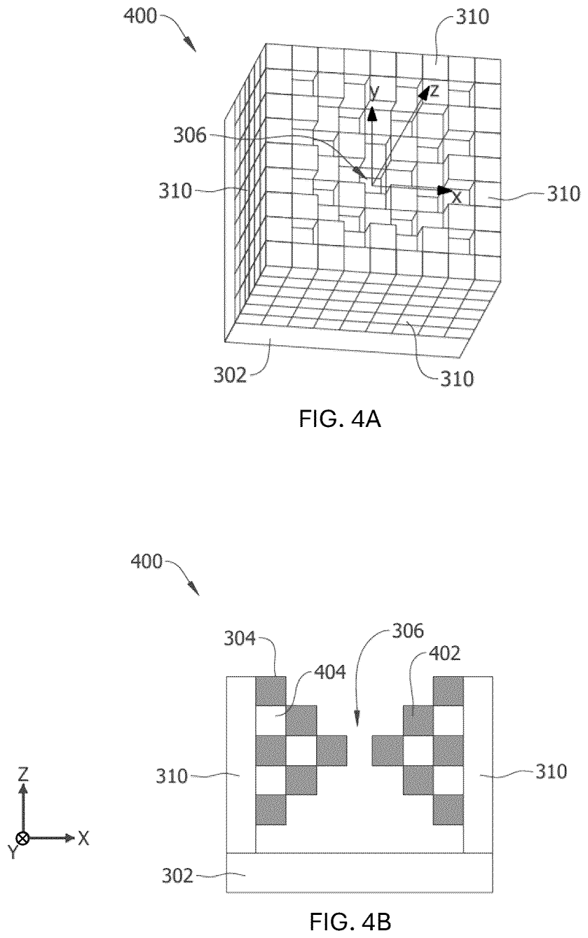

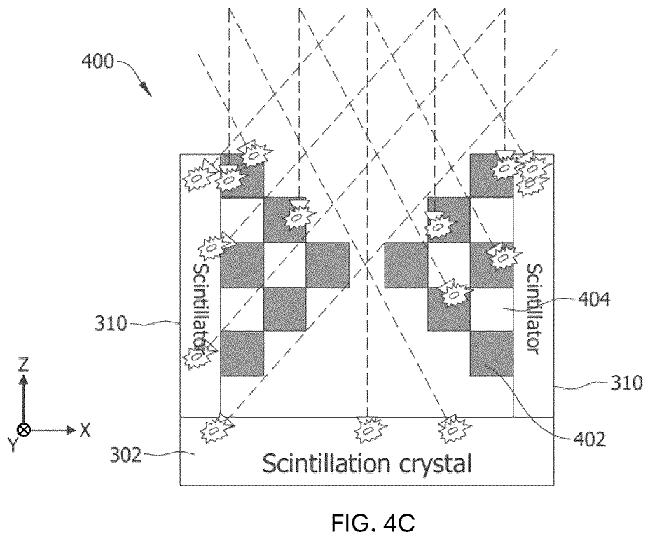

A more detailed embodiment of a BUGI module according to the present disclosure will now be discussed beginning with reference to FIGS. 4A and 4B and BUGI module 400. Similar components will be identified with the same reference numbers as the BUGI module 300 in FIG. 3 and they generally function similarly unless described otherwise.

FIG. 4A is an isometric view of BUGI module 400 and FIG. 4B is a simplified cross-sectional view of the BUGI module 400 taken along the X axis as viewed from the Y direction in FIG. 4A. FIG. 4C is the BUGI module 400 from FIG. 4B with the paths of example gamma photons shown as dashed arrows and indicating detection events by the scintillation crystals that are used to construct the module 400.

The BUGI module 400 replaces heavy metal (e.g., lead) with dense, inorganic scintillator material, thereby integrating the collimator as a part of the sensor, contributing more detected photons and hence higher sensitivity to an imaging system. In consideration of the complexity of manufacturing and signal readout, a simplified collimation geometry employing 3×3×3 mm crystal cubes is used. To achieve a preferred tradeoff between sensitivity and resolution, the example BUGI module employs a pinhole structure with checkerboard pattern, denoted as Coded Sensor. The main feature of this structure is that the lower layer of crystals is collimated by the crystal elements above it, hence each detected photon contains more directional information to decode the location of the source. In general, the design principles behind Coded Sensor Gamma Imager (CSGI) SPECT are: (a) to arrange the crystal cubes in alternating pattern using small active sensors 402 and non-scintillator cubes 404 (identified as non-shaded in FIG. 4B) to detect or encode gamma-rays as they pass through the coded sensor element that play the roles of pinhole aperture and or coded aperture; (b) to incorporate a bottom layer 302 of scintillator crystal that resembles the same geometry and functionality of a pinhole camera; (c) to include four side walls 310 of scintillator to provide information of coded aperture imaging based on the pattern of count distribution on side-wall detector surfaces.

As can be seen in FIG. 4C, a high percentage of photons whose trajectory intersects the BUGI module 400 will be detected. There are three general grouping for detection: 1) photons detected by the scintillation crystal layer 302, 2) photons detected by the coded sensor element 304, and 3) photons detected by the sidewalls 310. The photons whose trajectory passes through the aperture 306 of the coded sensor element 304 without interacting with the coded sensor element will reach the scintillation crystal layer 302 and be detected by the scintillation material in that layer, similar to the functioning of traditional gamma camera with a heavy metal pinhole collimator. Unlike, known traditional gamma cameras, photons whose trajectory intersects the coded sensor element will be either detected by the scintillation material in the coded sensor element itself, or will pass through (because of the alternation between photon transparent, non-scintillator material with the scintillation material) and strike and be detected by the scintillation material in the sidewall 310.

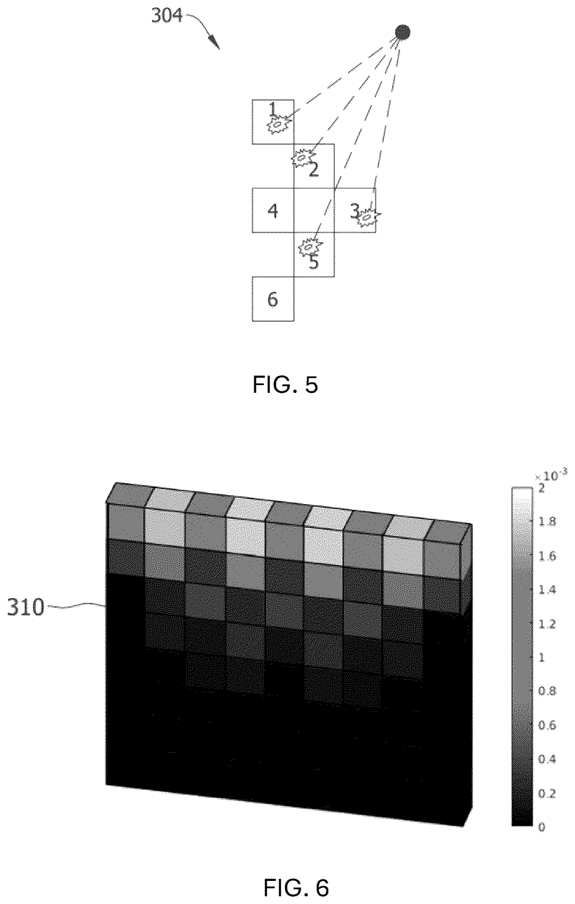

In addition to functioning as a pinhole camera style collimator for the scintillation crystal layer 302, the coded sensor element 304 itself functions as a detector/sensor. The alternating crystal structure encodes directional information in events detected by the scintillation crystals in the coded sensor element 304. FIG. 5 is a simplified view of six scintillation crystals (numbered 1-6) of a portion of the coded sensor element 304. In the example shown in FIG. 5, detection of a gamma ray by a scintillation crystal #5 is collimated by the detector elements above it (e.g., crystals 2 and 3). Therefore, photons detected by crystals at the lower layers of the coded sensor (e.g., #4-6) contain more directional information than those detected at the top layer (e.g., crystals 1-3).

The four side walls 310 providing coded aperture imaging in conjunction with the alternating structure of the coded sensor element 304 will be described with respect to FIG. 6. The alternating crystal structure in the coded sensor element and the detectors (e.g., the cuboid scintillation crystal material) in each of the 4 side walls function as a coded aperture camera. FIG. 6 shows the gamma-ray distribution on the detector surface of the upper sidewalls 310 (as viewed in FIG. 4A) when a Tc-99m point source is placed at (0, −25, 50) mm (assuming the origin is at the center of the coded sensor where the pinhole aperture is in FIG. 4A). This count distribution exhibits a coded aperture pattern defined by the alternating crystal structure in the coded sensor element 304.

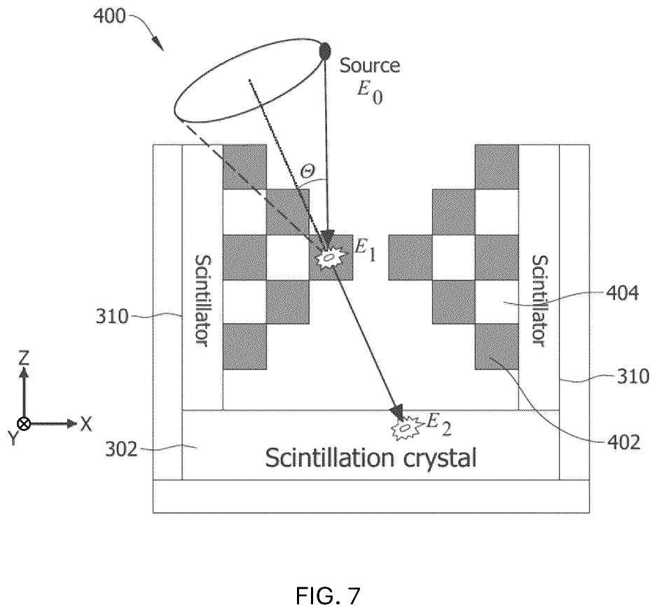

A further aspect of some embodiments is the functioning of the BUGI module as a Compton camera. FIG. 7 shows gamma-rays undergoing Compton scattering followed by photoelectric interaction in BUGI module 400, which can be utilized for Compton camera imaging.

Thus, a single BUGI module according to the present disclosure may provide the spatial and directional information from a conventional pinhole collimated gamma camera, a self-collimation camera, four coded-aperture cameras, and a Compton camera; resulting in substantially more counts and information content.

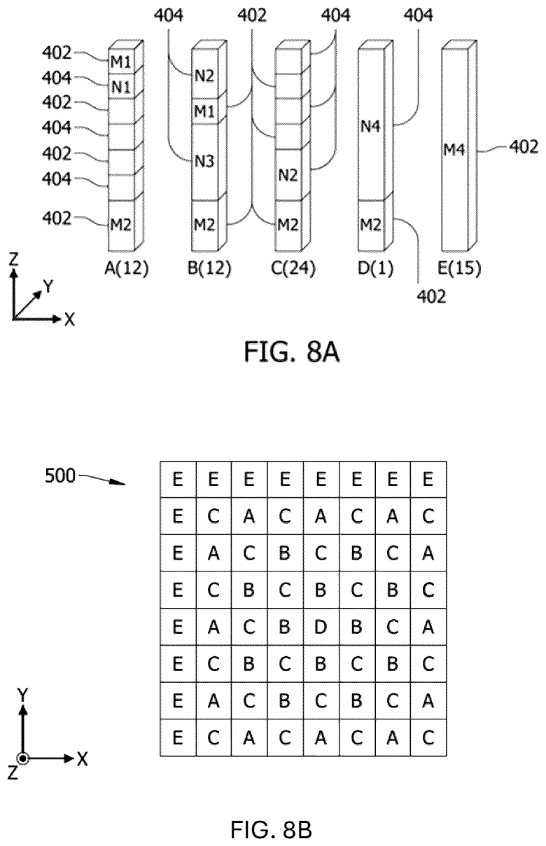

An example of the construction of an example BUGI module 500 will be described with reference to FIGS. 8A and 8B. Except as otherwise described, the BUGI module 500 is similar to the BUGI modules described above. The module 500 consists of five types of crystal columns denoted as A through E in FIG. 8A. These columns exhibit an alternating crystal pattern, comprised of seven types of segments with different dimensions listed in Table 1 below. Cuboids 402 represent GAGG scintillator material, and cuboids 404 are acrylic. The low density acrylic parts serve as both supporting structure and light guides that transport scintillation photons towards top and bottom ends. All crystals and acrylic surfaces are polished. In some embodiments, reflective films (ESR from 3M, 70 μm thick) are applied to the 4 long side surfaces of each detector column to provide optical isolation and to improve light extraction efficiency. Each crystal column is measured approximately 3 mm×3 mm×24 mm, forming an 8×8 crystal array shown in top view in FIG. 8B. The letters in FIG. 8B correspond to the column types in FIG. 8A. All these columns are arranged in a specific sequence to be shaped into a pinhole structure, with column D forming the pinhole aperture. Due to readout limitations, this example module was constructed with only two sidewalls, which are composed of columns of the type E.

TABLE 1

| Type | Size (mm) |
|---|---|
| M1 | 3 × 3 × 3 |
| M2 | 3 × 3 × 6 |
| M4 | 3 × 3 × 24 |
| N1 | 3 × 3 × 3 |
| N2 | 3 × 3 × 6 |
| N3 | 3 × 3 × 9 |
| N4 | 3 × 3 × 18 |

Material:
M = GAGG;
N = Acrylic

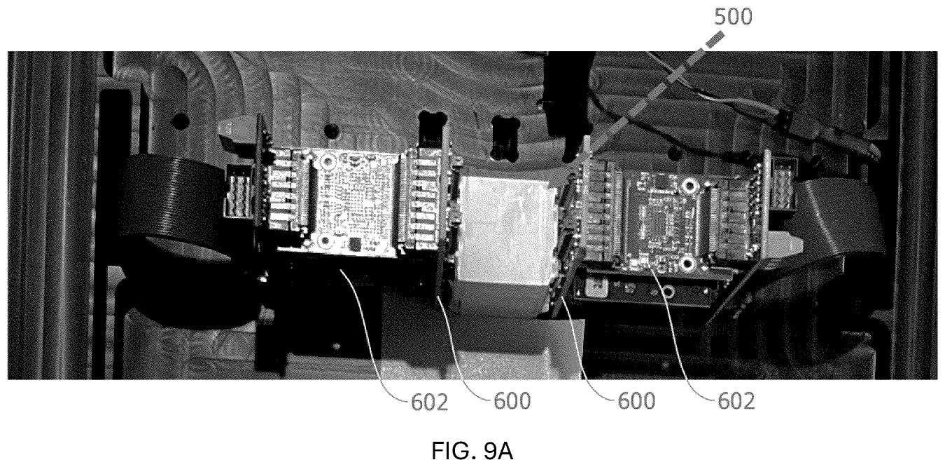

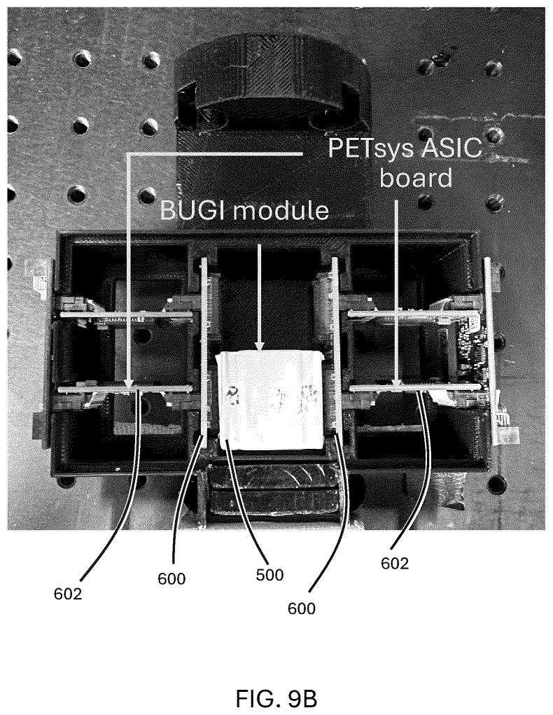

In an example constructed BUGI detector module, each crystal column is coupled in a 1:1 ratio to a silicon photomultiplier (SiPM) array 600 (S14161-3050HS-08, Hamamatsu Photonics, Japan) at the top and bottom (i.e., dual-ended readout), as shown in FIGS. 9A and 9B. A data acquisition system (DAQ) 602 using a TOFPET2 ASIC (PETSys Electronics, Portugal) enables independent readout of 64 SiPM channels. Scintillation light photons produced in each detector column are read out by two SiPM channels-one on top and one at the bottom. The sum of the 2 signals is proportional to the total energy deposited by the gamma-ray interaction. The location of a gamma-ray interaction within the detector column is determined by the ratio of the two SiPM signals according to (T−B)/(T+B), where T and B are the intensity of the top and bottom SiPM signal, respectively. The SiPM array 600 and the DAQ 602 collectively form the readout circuitry for the BUGI module. If all four sidewalls were included in the example BUGI module, the module would be a 9×9 crystal column array. However, due to constraints with the available 64 SiPM readout channels, two of the side walls were removed in this example embodiment. This adjustment does not compromise the functionality of the module which still provides the spatial and directional information from a conventional pinhole collimated gamma camera, a self-collimation camera, two coded-aperture cameras, and a Compton camera. Alternatively, any resulting asymmetry can be readily compensated by flipping the example BUGI module 180 degrees relative to the central axis of the pinhole aperture to acquire additional data from the missing side walls. In FIG. 9B, the module 500 and the readout circuitry are mounted in a prototype housing.

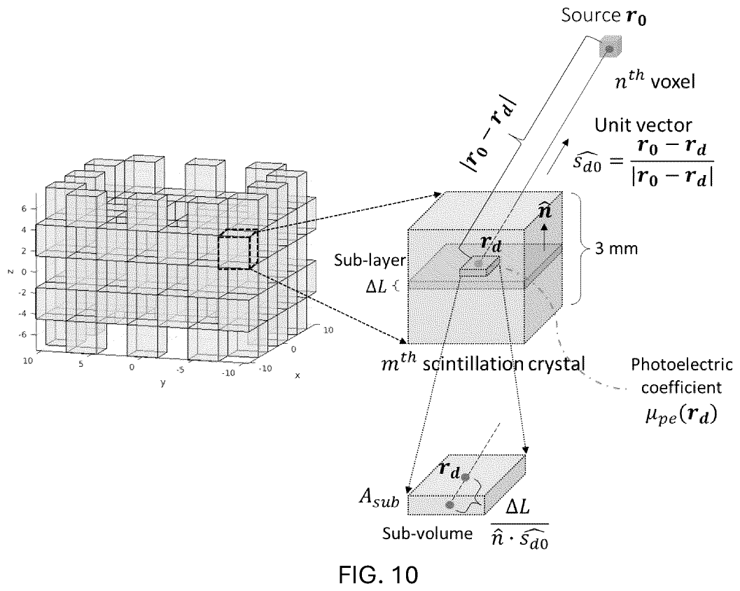

Unlike traditional pinhole collimation SPECT, the point spread function of the BUGI modules of this disclosure (including module 500) are not a simply blurred point-like distribution on a camera surface, but rather, shows more complicated pattern in a three-dimensional detector volume. Therefore, a new method for analytical derivation of the system response function is developed. FIG. 10 is an analytical model of system response function.

More specifically, an analytical radiative-transport equation method was developed to determine the system point spread function (PSF). This derivation shows the radiation flux detected by a single detector element due to an isotropic point source. Consider an isotropic point source located at the 3-D spatial location r₀ emitting photons at a constant rate λ₀. This source distribution can be described as a delta function

$$\frac{{λ}_{0}}{4 π} δ ( r - {r}_{0} ) .$$

Consider a location r_{d} in the m^{th} detector crystal. Using the Bolzman radiative transport equation, the photon distribution function ω(r_{d}, ŝ) at location r_{d} and in direction ŝ is given by:

$$ω ( {r}_{d} , \hat{s} ) = \frac{{λ}_{0}}{4 π {c}_{m}} \int δ ( {r}_{d} - {r}_{0} - \hat{s} l ) exp [ - {\int }_{0}^{l} {μ}_{tot} ( {r}_{d} - \hat{s} {l}^{′} ) d {l}^{′} ] dl \qquad ( 1 )$$

where cₘ denotes the speed of light in the medium and μₜₒₜ(r) denotes total attenuation coefficient at location r, which is a function of material through which the photon is traversing. Simplifying the above expression yields:

$$ω ( {r}_{d} , \hat{s} ) = \frac{{λ}_{0}}{4 π {c}_{m} \vert {r}_{d} - {r}_{0} {\vert}^{2}} \cdot exp [ - {\int }_{0}^{\vert {r}_{d} - {r}_{0} \vert} {μ}_{tot} ( {r}_{d} - \hat{s} {l}^{′} ) {dl}^{′} ] δ ( \hat{s} - \text{?} ) \qquad ( 2 )$$

where

$$\text{?} = \frac{{r}_{d} - {r}_{0}}{\vert {r}_{d} - {r}_{0} \vert} \qquad ( 3 )$$

To determine the radiant flux deposited in the m^{th} scintillation crystal, this crystal is divided into multiple thin layers of thickness ΔL. Consider that r_{d} is located in one of these layers and consider a small area of magnitude ΔA around r_{d}. Together this layer and area denote a sub-volume. The photon flux recorded within this small sub-volume due to the photon emission at location r₀ is given by:

$${φ}_{m , subVol} ( {r}_{0} ) = \int {\int }_{{A}_{s u b}} {c}_{m} ω ( {r}_{d} , \hat{s} ) ( \hat{n} \cdot \hat{s} ) \frac{{μ}_{p e} ( {r}_{d} ) Δ L}{\hat{n} \cdot \hat{s}} {d}^{2} {r}_{d} d \hat{s} \qquad ( 4 )$$

where \hat{n} is the norm vector of the thin slab, and μₚₑ(r_{d}) is the photoelectric coefficient. Simplifying further:

$${φ}_{m , subVol} ( {r}_{0} ) = \frac{{λ}_{0} Δ L}{4 π} \cdot {\int }_{{A}_{s u b}} \frac{{μ}_{p e} ( {r}_{d} )}{{\vert {r}_{d} - {r}_{0} \vert}^{2}} exp [ - {\int }_{0}^{\vert {r}_{d} - {r}_{0} \vert} {μ}_{tot} ( {r}_{d} - \text{?} {l}^{′} ) {dl}^{′} ] {d}^{2} {r}_{d} \qquad ( 5 )$$

Since this is a small volume, it can be assumed that the flux of photons does not change substantially as r_{d} varies. Thus, r_{d} is replaced by the center of the sub-volume r_{m,i}:

$${φ}_{m , i} ( {r}_{0} ) = \frac{{λ}_{0} {μ}_{p e} ( {r}_{m , i} ) Δ L Δ A}{4 π \vert {r}_{m , i} - {r}_{0} {\vert}^{2}} \cdot exp [ - {\int }_{0}^{\vert {r}_{m , i} - {r}_{0} \vert} {μ}_{tot} ( {r}_{m , i} - \text{?} {l}^{′} ) {dl}^{′} ] \qquad ( 6 )$$

where

$$\text{?} = \frac{{r}_{m , i} - {r}_{0}}{\vert {r}_{m , i} - {r}_{0} \vert} \qquad ( 7 )$$

where i denotes the sub-crystal index. Also note that the term ΔLΔA is the volume of the sub-crystal ΔV. The flux detected over the entire volume of the crystal is the summation of flux over each sub-volume, so that

$${φ}_{m} ( {r}_{0} ) = {\sum }_{i = 1}^{N} \frac{Δ V \cdot {λ}_{0} {μ}_{p e} ( {r}_{m , i} )}{4 π {\vert {r}_{m , o} - {r}_{0} \vert}^{2}} exp [ - {\int }_{0}^{\vert {r}_{m , i} - {r}_{0} \vert} {μ}_{tot} ( {r}_{m , i} - \text{?} {l}^{′} ) {dl}^{′} ] \qquad ( 8 )$$

where N denotes the total number of discrete sub-volumes. To obtain the system matrix, consider a voxelized object space. Denote the support of the n^{th} voxel is φₙ(r). Then the (m,n)^{th} element of the system matrix is given by:

$${H}_{m n} = \int {φ}_{m} ( {r}_{0} ) {ϕ}_{n} ( {r}_{0} ) {d}^{3} {r}_{0} \qquad ( 9 )$$

where φₙ(r) can be defined as below:

$${ϕ}_{n} ( r ) = \{ \begin{matrix}1 , & r lies within voxel n \\ 0 , & otherwise\end{matrix} \qquad ( 10 )$$

Substituting the expression for φₘ(r₀) from Eq. (8) yields

$${H}_{m n} = \int {d}^{3} {r}_{0} {ϕ}_{n} ( {r}_{0} ) \cdot {\sum }_{i = 1}^{N} \frac{Δ V \cdot {λ}_{0} {μ}_{p e} ( {r}_{m , i} )}{4 π {\vert {r}_{m , i} - {r}_{0} \vert}^{2}} exp [ - {\int }_{0}^{\vert {r}_{m , i} - {r}_{0} \vert} {μ}_{tot} ( {r}_{m , i} - \text{?} {l}^{′} ) {dl}^{′} ] \qquad ( 11 )$$

The object space is discretized into 51×51×51 cubic voxels, each measuring 2×2×2 mm³. The system matrix was pre-computed and stored for image reconstruction. For reconstruction process, the maximization likelihood expectation maximization (MLEM) algorithm was implemented.

In the example system 100, GPU-based list-mode PET reconstruction is employed. The coincidence events detected between the first and second detector modules are stored in list-mode format, then processed with a previously developed GPU-based list-mode maximum-likelihood expectation-maximization (MLEM) reconstruction framework, where the system matrix is computed on-the-fly based on a tube-of-response connecting a detector crystal in the first detector module 135 and a detector crystal in the second detector module 137 and a TOF Gaussian kernel. In one example embodiment, spatially invariant Point Spread Function (PSF) model is applied with a tube-of-response kernel width of 1.5 mm FWHM and the object space is divided into 200×200×150 cubic voxels, each voxel is 1×1×1 mm³ in size. Attenuation correction, random correction and scatter correction are available for the list-mode reconstruction framework but not applied for the imaging experiments below due to the small object size and low activity levels.

Monte Carlo (MC) simulations using GATE v9.2 were conducted to validate the analytical framework described by Eq. (11). In this study, the example BUGI detector module has the same geometry configuration as in FIG. 4A, which consists of 450 GAGG scintillator cuboids and 198 low-density plastic cuboids serving as lightguides. The energy resolution of GAGG scintillator is set as 10% full-width-at-half-maximum (FWHM) for 511 keV gamma-rays. Each cubic component measures 3 mm×3 mm×3 mm, and the overall dimensions of the detector module are 27 mm×27 mm×24 mm. To minimize impacts from photons outside the imaging field-of-view (FOV), all four side surfaces of the module are shielded with 6-mm-thick lead. This shielding design ensures that interactions in the four side-wall detectors are predominantly from photons that pass through and collimated by the spatial encoding sensor element (as opposed to photons entering the BUGI detector module from exterior surface of the side-wall detectors). With the center of the pinhole aperture defined as the origin of the coordinate system, a Tc-99m point source with an activity of 1 MBq is initially positioned at coordinates (0,0,150) mm above the detector and subsequently moved to coordinates (50, 50, 150) mm. Data acquisition is performed for 5 minutes at each location. Photoelectric events detected by each GAGG crystal are recorded using an energy window of 140 keV±10%. Correlation between the MC simulated event distribution and detection probability predicted using Eq. (11) was calculated for the two point-source locations above.

To further validate the system model, the sensitivity image of the SUPRIM system (which is needed for image reconstruction) estimated from the following MC MLEM simulation was compared with the analytical model in equation (11). A Tc-99m point source was moved across the XY plane above the pinhole aperture at Z=150 mm, with increments of 3 mm, within a 60 mm×60 mm imaging FOV. The activity of the point source was set as 10 kBq, with an acquisition time of 25 minutes for each location. By summing up photo-electric events detected from all GAGG components corresponding to each source location, a sensitivity image of plane Z=150 mm was generated for one BUGI detector module. This sensitivity image was then compared with the one estimated using the corresponding system matrix in equation (11), where the sensitivity value of each voxel n in the plane is

$${S}_{n} = {\sum }_{m = 1}^{M} {H}_{m n} \qquad ( 12 )$$

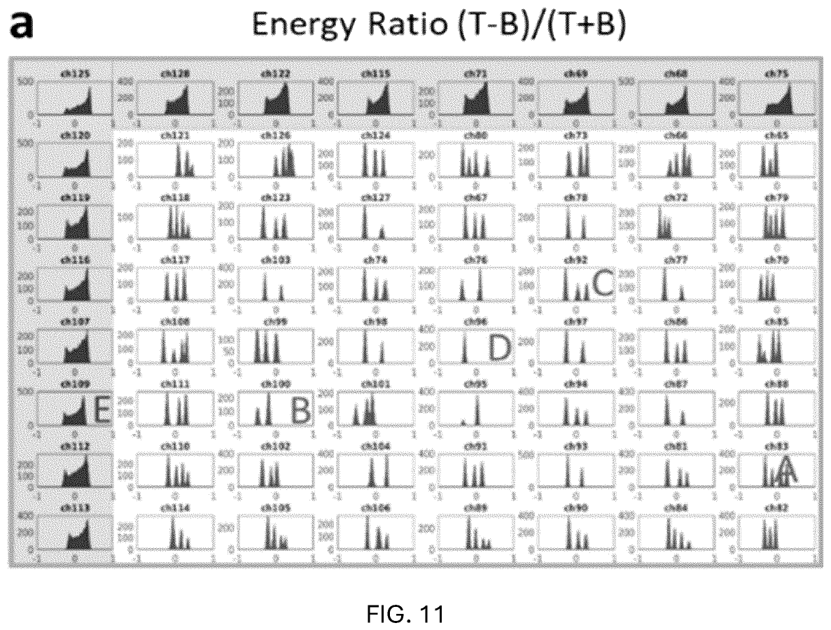

The preliminary tests of the BUGI module 500 using dual-ended readout scheme and TOFPET2 ASIC boards in FIG. 9B show the location of gamma ray interaction in the BUGI module 500 and the amount of energy deposition can be resolved successfully for a broad range of gamma ray energies from 70 keV to more than 511 keV. FIG. 11 is a map of histograms of energy ratio (defined by the difference between the top (T) and bottom (B) SiPM signal intensity divided by the total light collected: (T−B)/(T+B)) when the example BUGI module 500 in FIG. 9B was irradiated by a Na-22 source. Each GAGG segment contributes a peak to this histogram. For instance, column B comprises two scintillator segments, the energy ratio histogram will display two distinct peaks. The histograms are the distribution of event energy ratio measured by 2 SiPM for each of the 64 detector columns. Gamma rays detected by scintillation crystals 402 in a BUGI module form discreate peaks in the energy ratio histograms because the scintillation light photons are originated from a discrete scintillator volume that is sandwiched by adjacent acrylic elements to create unique optical transport patterns for each coded sensor element 402. As a result, gamma ray interaction point in a BUGI module can be clearly identified. The side-wall detectors are made of 24 mm long GAGG crystals, showing a continuous and broad event distribution in the ratio plots. These ratio plots are used to calibrate the depth-of-interaction (DOI) of a gamma ray as a function of the SiPM signal ratio to create a lookup table for subsequent imaging experiment. In FIG. 11, the different types of columns discussed above are identified by the same corresponding letters “A”-“E”. After re-sorting events based on DOI classifier, the energy spectra of each cuboids show better energy resolution of ~10% for 511 keV photopeak.

Additional details of BUGI detector modules that may be used in the systems of the present disclosure may be found in International Application No. PCT/US20205/038121, which is incorporated herein by reference in its entirety for all purposes. Although generally described herein as including two SiPM arrays, some embodiments of the BUGI detector modules include more or fewer than two SiPM arrays.

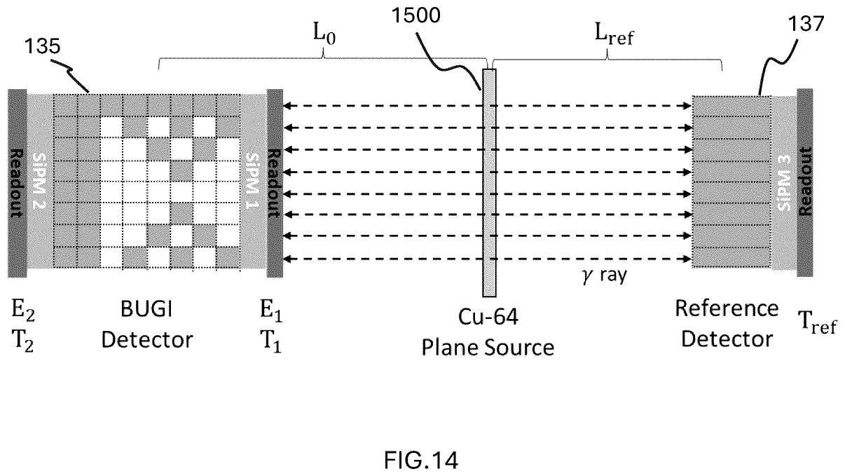

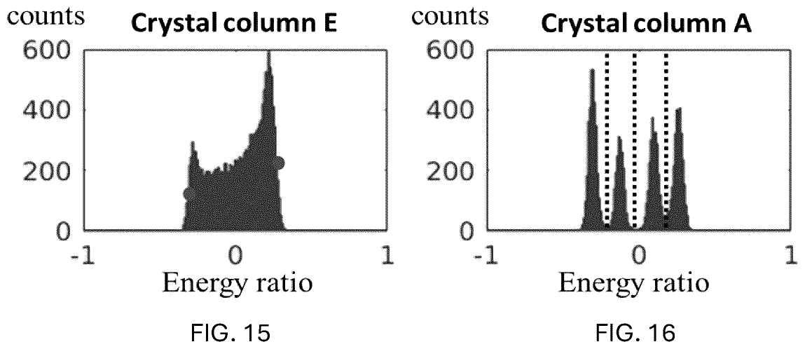

In order to perform SPECT and PET imaging, the first and second detectors 135, 137 of the system 100 need to be calibrated. FIG. 14 is the setup for detector calibration for a system using one BUGI detector module as the first detector module 135 and one TOF-PET detector array as the second detector module. However, the techniques for calibration may also be applied to systems using two or more BUGI detector modules or other combinations of types of detector modules. The calibration includes DOI calibration, crystal identification, energy lookup table, and depth-dependent timing alignment. A plane source 1500 is placed in between the BUGI detector module (the first detector module 135) and a reference PET detector module (the second detector module 137) comprised of 8×8 LYSO crystal array and a SiPM 3 array. The size of each LYSO crystal is 3.12×3.12×10 mm. Coincidence events recorded between SiPM 1 and SiPM 2 are used for DOI calibration and energy determination of the BUGI detector. Two DOI discrimination strategies were applied to different types of crystal columns in BUGI module, as they exhibit different distributions in the histogram plot of the ratio of top and bottom SiPM signals from individual events as shown in FIGS. 15 and 16. For example, column E in the BUGI detector module is a long single piece of scintillator, resulting in a continuously distributed histogram when plotting the ratio of

$$\frac{{E}_{1} + {E}_{2}}{{E}_{1} + {E}_{2}} .$$

The half maximum value of the falling edges of two peaks are assumed to correspond to the top and bottom surfaces of the crystal column (marked as red dots in FIG. 15). The DOI information is then estimated with the following equation:

$$DOI = k \cdot \frac{{E}_{1} - {E}_{2}}{{E}_{1} + {E}_{2}} + b \qquad ( 13 )$$

where E₁ and E₂ represent the signal intensity recorded by SiPM 1 and SiPM 2, k and b are obtained from linear fitting of the two data points. On the other hand, column A consists of four segmented GAGG crystals, contributing to four distinct peaks shown in the graph in FIG. 16. Since these peaks are well separated, the DOI can be directly determined based on the calibrated windows corresponding to individual crystal elements in the ratio histogram. In some other embodiments, DOI information and energy determination is estimated using double coincidence events with any two photon sensor elements of the BUGI detector module, including two elements of the same SiPM. In some embodiments, the DOI information and energy information is estimated in a BUGI detector with only a single SiPM.

In the next step, triple coincidence events among SiPM 1, SiPM 2 and SiPM 3 are used for timing calibration. Note that lines of response (LORs) at oblique angles in FIG. 14 are discarded to ensure that only head-on gamma-rays are considered for timing alignment. Assume that the time stamps for an event recorded at a specific depth L₀ from the source are T₁, T₂ and T_{ref}, the timing correction values T_{offset1} and T_{offset2} follow the equations:

$${T}_{ref} - ( {T}_{1} - {T}_{offset 1} ) = \frac{{L}_{ref} - {L}_{0}}{c} \qquad ( 14 )$$

$${T}_{ref} - ( {T}_{2} - {T}_{offset 2} ) = \frac{{L}_{ref} - {L}_{0}}{c} \qquad ( 15 )$$

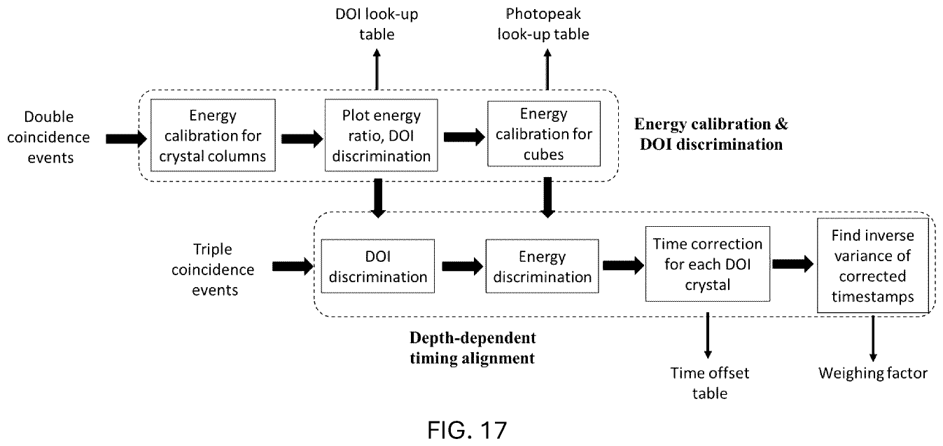

Then the trigger time of this event can be derived based on a weighted average of corrected time stamps T₁−T_{offset1} and T₂−T_{offset2} given by:

$${T}_{0} = {ω}_{1} \cdot ( {T}_{1} - {T}_{offset 1} ) + {ω}_{2} \cdot ( {T}_{2} - {T}_{offset 2} ) \qquad ( 16 )$$

where ω₁ and ω₂ are weighting factors, defined as inverse of variance of corrected trigger times at both ends. The whole framework is illustrated as in FIG. 17. In some embodiments, the BUGI module the timing calibration is performed using double coincidence events detected by SiPM3 and one of SiPM1 or SiPM2. In some such embodiments, the BUGI module includes only one of SiPM1 or SiPM2.

The example BUGI detector module shown in FIGS. 9A and 9B and discussed above was calibrated as described above and studied in connection with a TOF-PET detector array as the first detector module 135 and the second detector module 137 respectively.

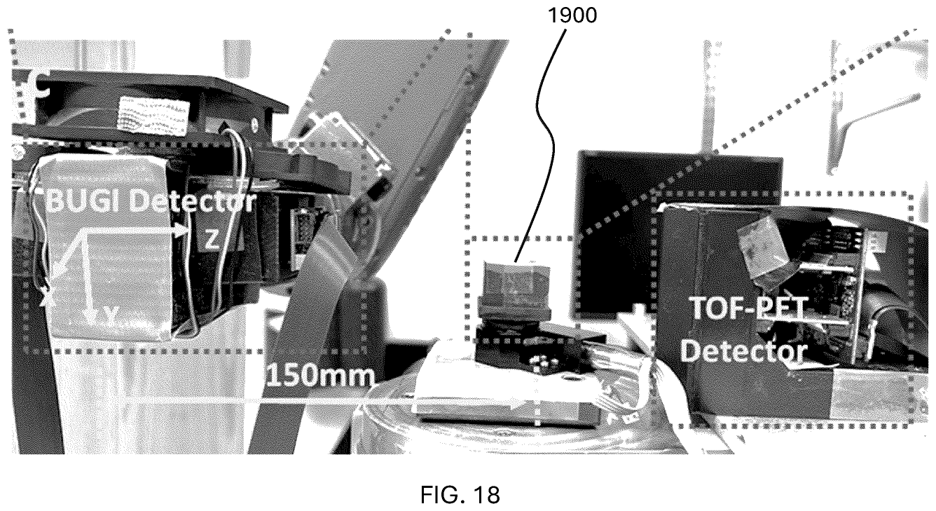

The BUGI module 500, SiPM arrays 600, and PETsys ASIC boards 602 were enclosed within a custom 3D-printed light-tight holder. The top of the holder was mounted to a KUKA robot arm for defined acquisition locations. Two 5V cooling fans were attached to the side surface of the holder for temperature control. A panel with a 2×2 TOF-PET detector arrays is positioned on the opposite side of BUGI module. Each detector array contains 8×8 LYSO crystal elements, with each crystal measuring 3.12×3.12×10 mm. Similar to the BUGI detector module, the LYSO crystals are separated by ESR film. Signals from both BUGI and LYSO detector arrays were acquired using PETsys electronics. This setup is shown in FIG. 18.

First, SPECT imaging using the BUGI module 500 was compared with a known clinical SPECT scanner. Many alpha-emitting radionuclides are associated with higher energy gamma rays (e.g. 440 keV emission from Ac-225 decay chain, 510.8 keV and 583 keV gamma rays from the daughter of Pb-212) than diagnostic radioisotopes. Therefore, Cu-64, a positron emitter with 17.5% branching ratio, was used to evaluate the imaging capability of a BUGI module against a conventional SPECT scanner.

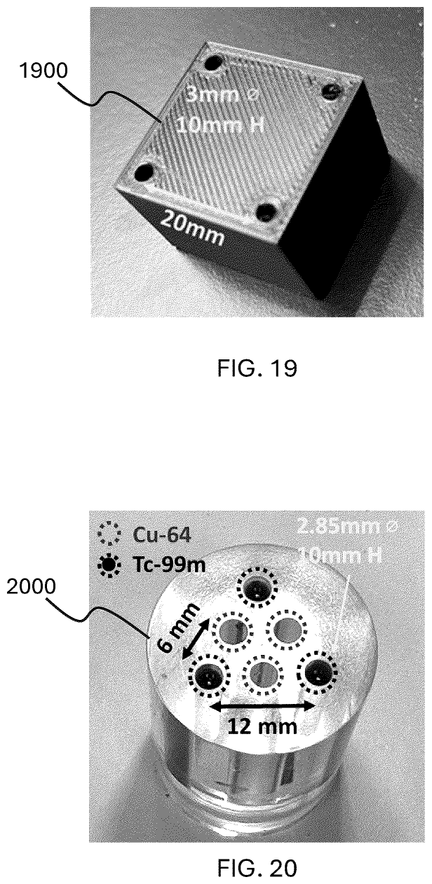

FIG. 19 is an image of a hot-rod phantom 1900 used for these first phantom studies. The phantom 1900 contains 4 chambers of 10 (height)×3 (0) mm rods and was filled with 0.12 mCi of Cu-64 solution each. The phantom was first imaged on a clinical dual-head SPECT scanner (Siemens Symbia Intevo) with a high-energy general purpose (HEGP) collimator following a standard-of-care (SOC) body imaging protocol. The total number of view-angles was 64, with an acquisition time of 30 seconds per angle. Subsequently, the same phantom was placed 15 cm away from the pinhole aperture of the BUGI module 500, as shown in FIG. 18. The phantom was rotated incrementally in 7 steps from 0° to 180°. The total acquisition time was equivalent to 32 minutes, same as the clinical imaging protocol after the radioactive decay of the source was accounted for. Detailed information of the comparison study is listed in Table 2 below

TABLE 2

| | | Clinical SPECT | |
|---|---|---|---|
| | | (Siemens Symbia | |
| Scanner | BUGI | Intevo) | |
| Collimation | N/A | High-energy | |
| | | general purpose | |
| | | (HEGP) collimator | |
| Number of detectors | 1 | 2 | |
| Distance to phantom | 150 mm | 250 | mm |
| Acquisition | 32 min | 32 | min |

| | | |
|---|---|---|
| time | equivalent | |
| Size of | 25 mm × 25 | ~600 mm × 400 mm |
| detector | mm × 24 mm | |
| Number of views | 7 | 64 |
| Voxel size | 2 mm × 2 | 2.4 mm × 2.4 |
| | mm × 4 mm | mm × 2.4 mm |

During the SPECT imaging described above, coincidence events between the BUGI detector module 500 and the reference TOF-PET detector were recorded. DOI and TOF information were incorporated in a PET image reconstruction process.

Simultaneous PET-SPECT imaging was conducted with a cylindrical phantom 2000 shown in FIG. 20. The phantom 2000 was 25 mm Ø and 40 mm tall containing 6 fillable rods of 2.8 mm Ø and 30 mm deep. The imaging was conducted using the same setup in FIG. 18, but with the phantom 2000 in place of the phantom 1900. The three outer rods were filled with ~30 μCi of Tc-99m each. The three inner rods were each filled with ~100 μCi of Cu-64. Data was acquired for 5 minutes each from 12 different angles covering from 0° to 330°.

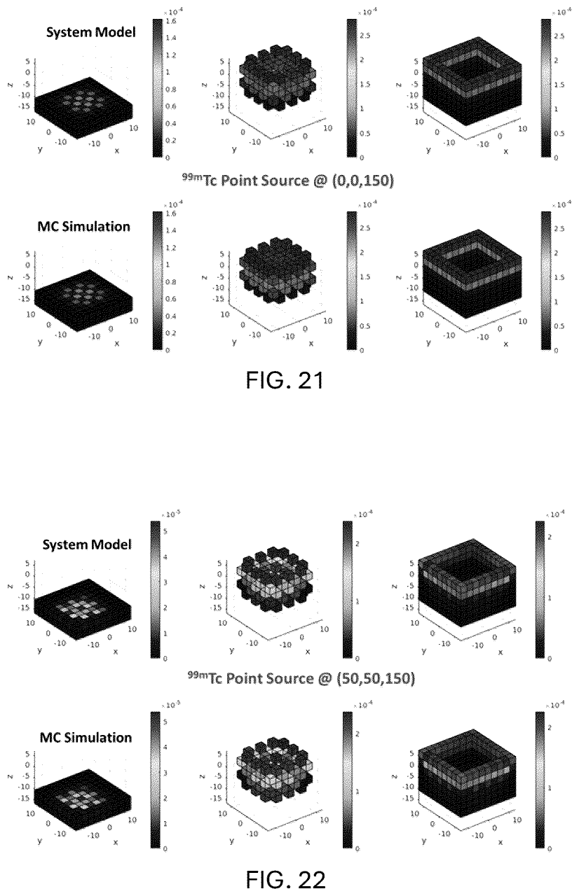

As a first step of validation, the distributions of detected counts obtained from the forward model outlined in equation (11) was compared with those generated through Monte Carlo simulation. FIGS. 21 and 22 are two representative count distributions from three parts of detector module corresponding to source locations at (0,0,150) mm and (50,50,150) mm respectively. In FIG. 21, the bottom camera displays a highlighted region in the center, whereas in FIG. 22, the projection shifts in the opposite direction relative to the source movement, which is in accordance with a conventional pinhole gamma camera. For the spatial encoding sensors and side walls, a majority of photons will be stopped by top layers when the incident angle is small. Yet the bottom layers receive more counts as the incident angle increases.

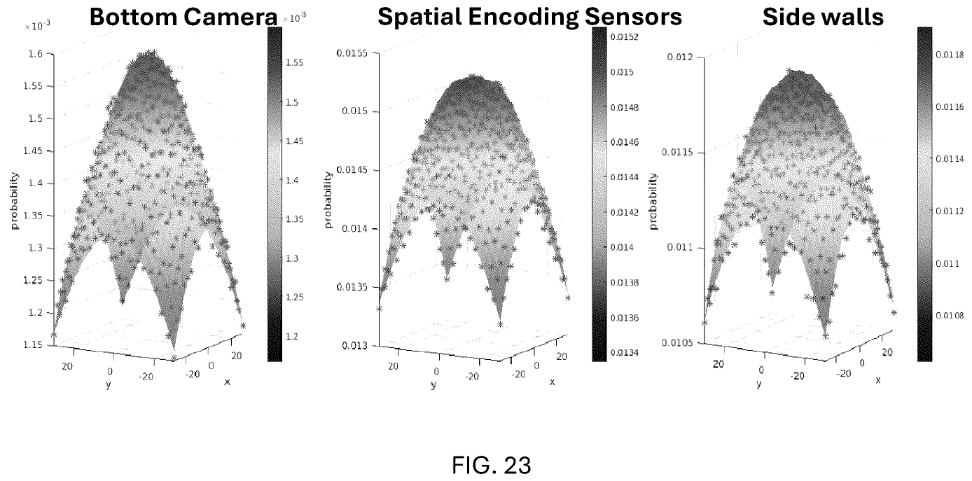

The sensitivity images of bottom camera, spatial encoding sensors and side walls are visualized as surface plots shown in FIG. 23. The object space has 51×51× 51 voxels, and each voxel is 2 mm×2 mm×2 mm in size. The MC simulation results, indicated by “*”, are overlaid onto the surface plots with a grid size of 8 mm. Note that the total counts detected by three types of detectors in the BUGI detector module from MC simulation were normalized based on the number of decays to calculate the probability. The surface plots match well with MC simulation data points, providing further validation of the system model in Eq. (11).

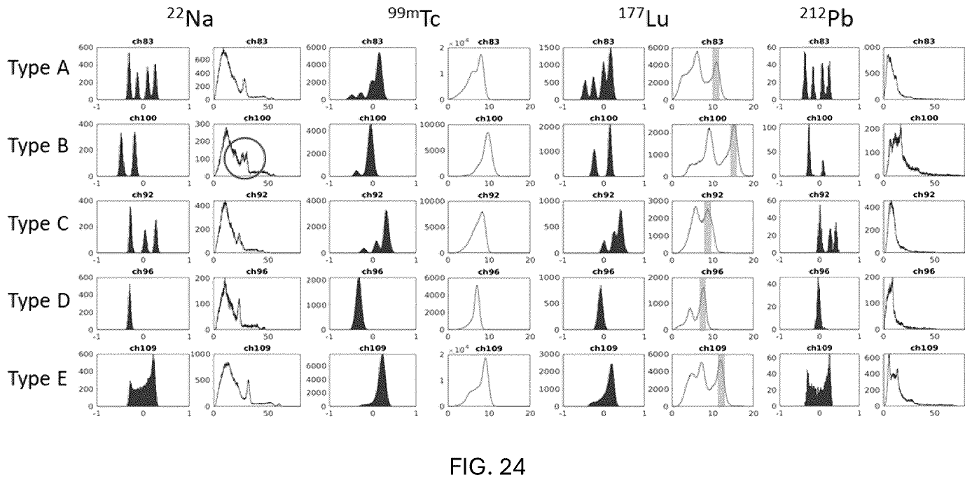

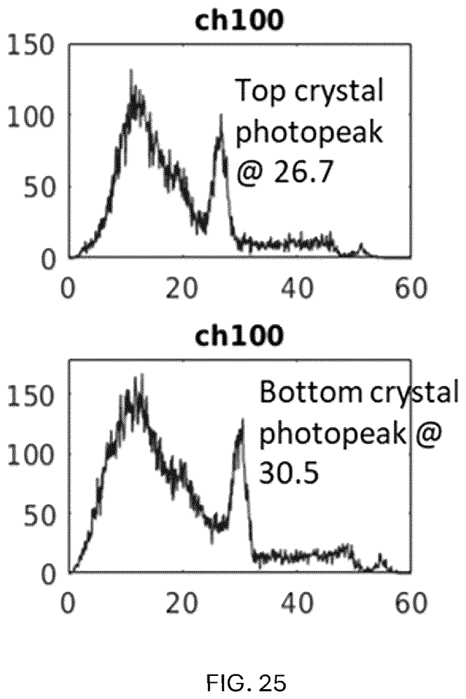

The spectroscopic performance was characterized with radionuclides that emit γ-rays across a broad range of energies, including 140 keV from Tc-99m, 113 keV or 208 keV from Lu-177, 511 keV and 1.27 MeV from Na-22, and Pb-212 that has a complex spectrum. Five representative channels—83, 100, 92, 96, and 109 coupled to crystal types A through E were selected to show the energy ratio histograms and energy spectra in FIG. 24. Distinct peaks are observed in the histograms of different crystal columns: four peaks in column A, three in column C, two in column B, and a single peak in column D. For low energy gamma rays, such as 140 keV emission from Tc-99m and 208 keV from Lu-177, the height of individual peaks in the energy ratio histograms exhibits significant nonuniformity due to the attenuation of gamma photons as they propagate through the scintillator material. In contrast, the height of peaks is less dependent on the depth of the GAGG crystal in the energy ratio histogram of higher energy emissions from Na-22 and Pb-212. Some crystal columns show multiple photoelectric peaks in energy spectra (e.g., as circled in FIG. 24), suggesting nonuniform light yield and/or light collection efficiency among multiple GAGG segments in a column. All the coincidence events were reprocessed based on the DOI classifier to identify where each gamma ray interacts. Events from multi-photoelectric peaks are now re-allocated to two distinct segments, resulting in reduced FWHM of the peaks shown in FIG. 25.

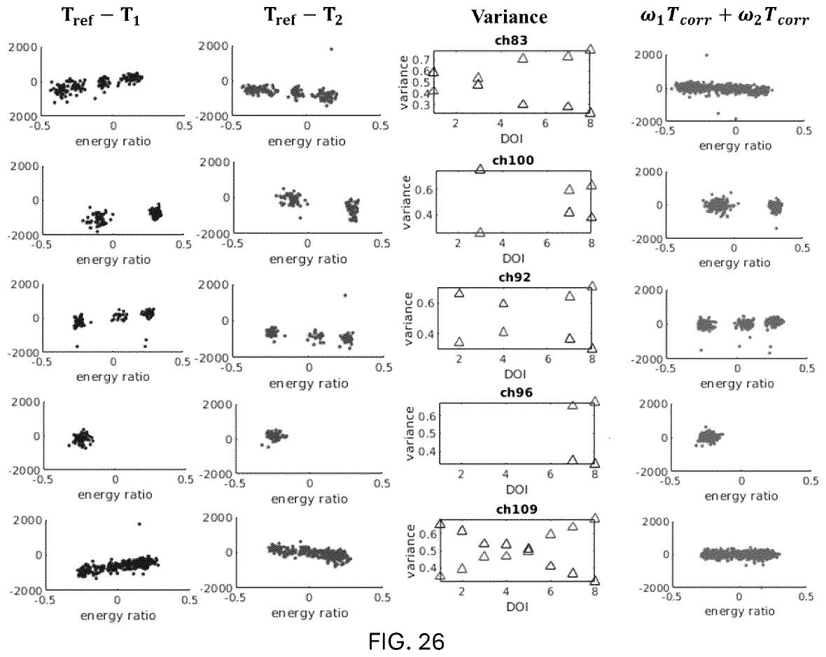

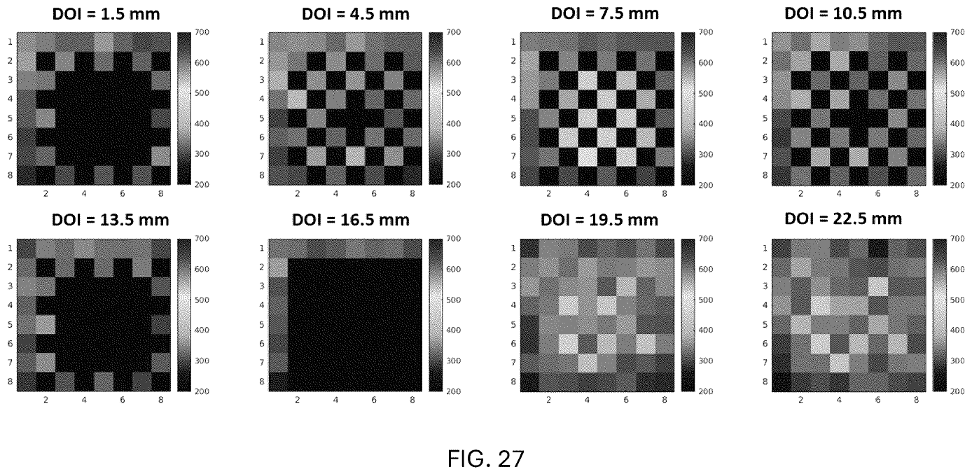

FIG. 26 includes graphs related to timing calibration from studying the system shown in FIG. 18. The first two columns show these timing differences plotted against the energy ratio. In the first column, where timing is measured relative to SiPM 1 (FIG. 14), the clusters become narrower as interactions move closer to the top end (i.e., left to right along the energy ratio axis), indicating reduced uncertainty due to more efficient light collection by SiPM 1 when gamma-ray interactions are near the top end. Conversely, the second column, relative to SiPM 2 (FIG. 14), shows increasing timing spread as interactions occur farther from the top end. Using these trends, time offsets for both SiPMs were extracted and timing variance across DOI bins was computed. These variances were used to derive weighting factors-defined as the inverse of variance (third column)-which were applied to emphasize the more temporally precise interaction time. The interaction time T₀ calculated as a weighted sum of the corrected time stamps, is shown in the fourth column. The resulting T₀ values remain flat and centered around zero across all DOI bins, indicating successful compensation for depth-dependent timing variation. The 2D distribution of the FWHM of the timing spectrum for each crystal element at eight DOI layers is shown in FIG. 27. These maps reveal consistent timing behavior across most of the detector area, with an average timing resolution of ~330 ps FWHM.

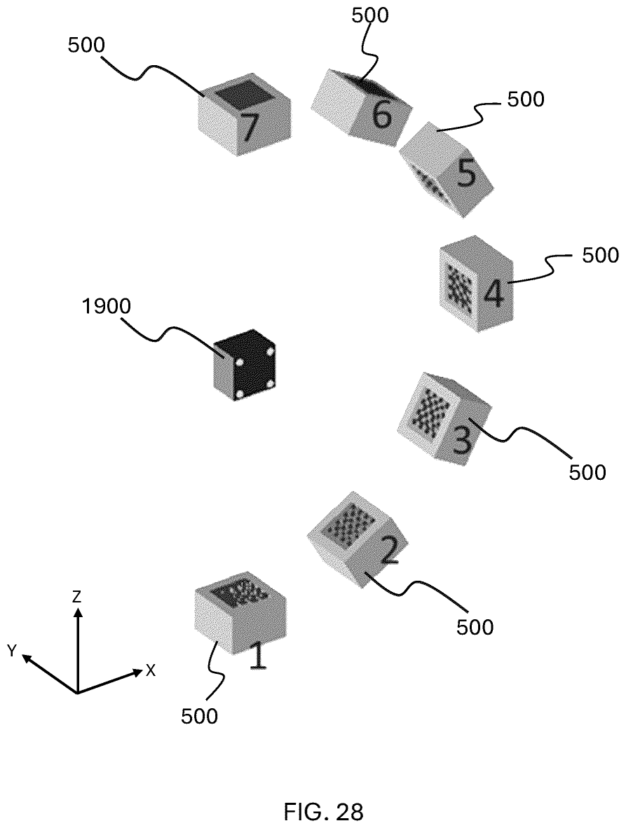

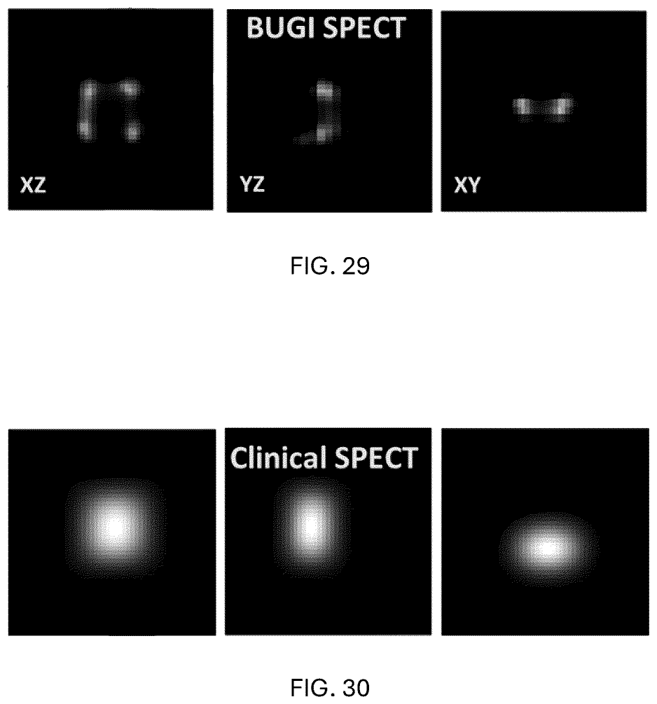

To test SPECT imaging, data acquisition protocol was a setup emulated to a multi-view system geometry depicted in FIG. 28, which may be referred to as a BUGI SPECT system to distinguish from the standard clinical SPECT system. In this configuration, the phantom 1900 was rotated at 7 angles, which is equivalent to a static system with seven BUGI detector modules 500 positioned around it to capture data. FIG. 29 shows the 3D image reconstruction result from BUGI SPECT. The rods on the right half are better resolved than those on the left, due to sampling from 0° to 180°. Nevertheless, these SPECT images demonstrate delineation of all rod sources and resolve the heterogeneous activity distribution in the phantom, which is completely lost in the clinical SPECT images shown in FIG. 30. More importantly, the number of photopeak events detected by the BUGI SPECT system exceeds that of a full clinical dual-head SPECT camera, with 3.7 million events compared to 2.16 million. This is achieved despite the BUGI module having only approximately 1/400th of the surface area of the clinical scanner, highlighting its superior sensitivity.

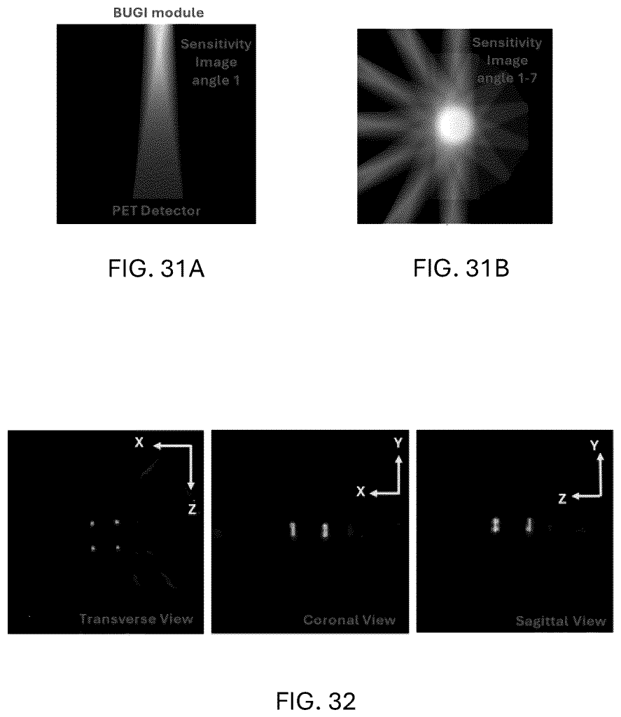

The sensitivity image of the test setup in FIG. 18 for PET imaging using the BUGI module prototype in coincidence with a TOF PET Detector is shown in FIGS. 31A and 31B when imaging from a single angle and 7 angles in 30° step increments, respectively. FIG. 32 shows PET images of the phantom 1900 in transverse, coronal and sagittal views, respectively. All 4 rod sources are clearly resolved. The resolution of the BUGI PET images is substantially higher than the BUGI SPECT images that are reconstructed using 511 keV gamma rays in singles mode without a conventional metal collimator. This marks the first simultaneously acquired SPECT and PET images (of a positron-emitting source) using the BUGI technology, demonstrating its superior imaging capability than conventional SPECT camera (in terms of system sensitivity) and an excellent PET image quality similar to typical TOF-PET scanners.

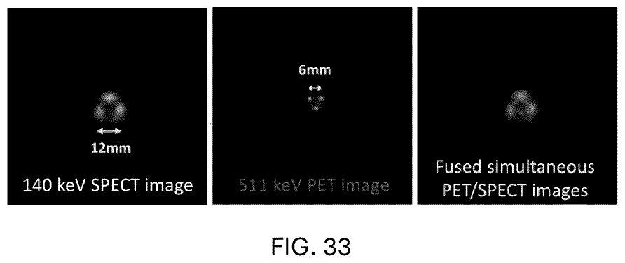

Simultaneous dual-isotope PET-SPECT imaging results are shown in FIG. 33. The images show the BUGI detection module successfully detected the heterogeneous distribution of 2 radionuclides in a mouse-size cylindrical phantom from the simultaneously acquired SPECT and PET images. The resolution of SPECT and PET are both adequate for organ level distribution in mice and human.

Design Optimization of BUGI Detector module. The example BUGI detector modules discussed above incorporate a thoughtfully combination of pinhole and coded aperture geometries to maximize the information content that each photon carries. Other embodiments include one or more of several variations that may enhance system performance: (1) Adjustments to crystal element size, the focal length and magnification factor of the pinhole aperture may further optimize the trade-off between field of view, resolution, and sensitivity. (2) Alternative geometry for the spatial encoding sensor element may increase the encoded spatial information to further improve the reconstructed SPECT images. (3) The example BUGI detector modules were constructed using GAGG-HL which is formulated for high light yield and better energy resolution-attributes that are well-suited for SPECT imaging, particularly in the context of αRPT where precise energy discrimination is critical. However, alternative materials such as GAGG-F or GSO, which offer shorter decay times, may improve timing resolution and could be advantageous for TOF-PET applications. As such, a careful balance must be considered depending on the imaging priority. Monte Carlo simulations can be employed to evaluate system performance across different scintillator choices. (4) The dual-ended readout enables DOI measurement but adds to the electronics complexity and system size. Transitioning to a single-ended readout with controlled scintillation light distribution such as the use of prism light guide on top of crystal columns will allow the BUGI detector module to be positioned closer to an object for even higher sensitivity. Another embodiment arranges the dual readout electronics horizontally to the side walls of the BUGI detector module and could achieve similar compactness.

Correction of system model with experimental data. While the system response model described above and used in BUGI SPECT was validated through Monte Carlo simulation, discrepancies can still arise in experimental settings. Two primary sources of mismatch include variation in detection efficiency across individual crystal elements, and the presence of SiPM arrays and readout electronics in the gamma ray path due to the dual-ended readout design. These components introduce additional attenuation and scattering effects that are not captured in the system response function modelling. Experimental correction using data acquired from a uniform plane source may be applied to account for these factors. This may allow normalizing crystal-to-crystal response variations and empirically compensate for unmodeled attenuation caused by readout hardware.

Timing alignment. In the examples discussed above, a one-time plane source acquisition approach was used instead of point source scanning, significantly reducing experimental effort and speeding up the calibration process. To minimize parallax error and ensure accurate measurement of time differences corresponding to known distance differences, only head-on coincidence events were considered. The example embodiment BUGI detector module achieve 330 ps FWHM timing resolution when calibrated against a reference TOF-PET detector consisted of 10 mm-thick LYSO crystals without DOI capability. Further enhancements in timing resolution are expected in embodiments using a symmetric pair of BUGI detector modules each contains short segments of GAGG crystals.

Down-scatter. Using the prototype system, 3D BUGI SPECT images of Cu-64 and Tc-99m were successfully reconstructed using system matrix based on single-energy gamma emissions. However, in the context of αRPT imaging, higher-energy gamma-rays from αRPT source can significantly overlap with 511 keV photons from PET tracer, leading to crosstalk contamination between isotopes. To address this challenge, some embodiments incorporate data from multiple energy windows into the reconstruction process. Joint reconstruction methods with model-based crosstalk compensation have shown potential to improve quantitative accuracy and increase effective system sensitivity. Implementation of such strategies includes an extension of the system model to account for photon interactions across multiple energy windows.

The example BUGI detector modules and systems described herein enable simultaneous PET and SPECT imaging. In a more specific embodiment, they enable ultra-sensitive SPECT imaging of αRPT at extremely low count levels, alongside PET imaging of DNA repair processes. Leveraging a multifunctional geometric design-including multi-pinhole collimation, coded aperture imaging, and self-collimation—the BUGI system achieves orders-of-magnitude higher sensitivity than conventional SPECT systems. Detector calibration demonstrated robust spectroscopic and temporal performance, with an average energy resolution of 10% at 511 keV and timing resolution of approximately 330 ps. The system is capable of detecting gamma emissions across a wide energy range (70 keV to 511 keV and above) and shows promising potential for TOF-PET applications. The system performance was successfully evaluated through experimental phantom studies. Cu-64 phantom imaging was successfully reconstructed using two independent frameworks for PET and SPECT, showing the detector's dual-modality capabilities. Compared to a clinical SPECT scanner, the BUGI detector module achieved higher spatial resolution and detected more photopeak events-despite operating with only 1/400th of the active detection area. In addition, simultaneous SPECT/PET imaging with Tc-99m and Cu-64 was demonstrated, achieving SPECT spatial resolution better than 12 mm FWHM and PET spatial resolution better than 3 mm FWHM.

This written description uses examples to disclose the invention, including the best mode, and also to enable any person skilled in the art to practice the invention, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

As used herein, the terms “about,” “substantially,” “essentially” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A dual modality imaging system for positron emission tomography (PET) and single-photon emission computed tomography (SPECT), the system comprising:
a first detector module configured to detect gamma photons for SPECT imaging of a subject from a first position proximate the subject without using a heavy-metal collimator and to generate first output data in response to gamma photons detected by the first detector module;
a second detector module configured to detect gamma photons from a second position proximate the subject opposite the first position and to generate second output data in response to gamma photons detected by the second detector module; and
a computer device in communication with the first detector module and the second detector module, wherein the computer device comprises at least one processor in communication with at least one memory device, wherein the at least one processor is programmed to:
receive the first output data from the first detector module;
receive the second output data from the second detector module;
perform SPECT imaging of the subject using the received first output data; and
perform PET imaging of the subject using coincidence data from the received first output data and the received second output data.

2. The dual modality imaging system of claim 1, wherein the first detector comprises a coded sensor gamma imager module including:
a scintillation crystal layer for detecting photons; and
a spatial-encoding sensor element positioned above the scintillation crystal layer, the spatial-encoding sensor element comprising scintillator material arranged to form a nonuniform structure in at least one dimension above the scintillation crystal layer providing coded-aperture functionality.

3. The dual modality imaging system of claim 2, wherein the scintillator material is arranged to form a pinhole geometry having an interior pinhole aperture above the scintillation crystal layer.

4. The dual modality imaging system of claim 3, wherein the scintillation material and the scintillation crystal layer comprise gadolinium aluminum gallium garnet (GAGG) scintillation crystals.

5. The dual modality imaging system of claim 2, wherein the second detector comprises an additional coded sensor gamma imager module.

6. The dual modality imaging system of claim 5, wherein the at least one processor is further programmed to perform SPECT imaging of the subject using the received second output data from the second detector module.

7. The dual modality imaging system of claim 2, wherein the second detector comprises a time-of-flight positron emission tomography (TOF-PET) detector.

8. The dual modality imaging system of claim 1, wherein the processor is programmed to perform SPECT imaging and PET imaging substantially simultaneously.

9. A method for positron emission tomography (PET) and single-photon emission computed tomography (SPECT) imaging comprising:
positioning a first detector module to receive first gamma photons from a subject, the first detector module comprising a coded sensor gamma imager module including a spatial-encoding sensor element comprising scintillator material arranged to have a nonuniform structure in at least one dimension and a scintillation crystal layer positioned below the spatial-encoding sensor element;
positioning a second detector module to receive second gamma photons from the subject;
collimating the received first gamma photons using the spatial-encoding sensor element;
detecting the first gamma photons by the spatial-encoding sensor element or by the scintillation crystal layer after the gamma photons pass through the spatial-encoding sensor element;
outputting, to a computing device, first data on the first gamma photons that are detected by the first detector module;
detecting the second gamma photons with the second detector module;
outputting, to the computing device, second data on the second gamma photons that are detected by the second detector module, the second data being synchronized with the first data; and
one or both of:
reconstructing, by the computing device, a SPECT image based at least in part on the first data; and
reconstructing, by the computing device, a PET image based at least in part on the first data and the second data.

10. The method of claim 9, wherein the second detector module comprises a time-of-flight positron emission tomography (TOF-PET) detector crystal array and detecting the second gamma photons with the second detector module comprises detecting the second gamma photons that interact with the TOF-PET detector crystal array.

11. The method of claim 9, wherein the second detector module comprises an additional coded sensor gamma imager module, and detecting the second gamma photons with the second detector module comprises detecting the second gamma photons by the spatial-encoding sensor element or by the scintillation crystal layer after the second gamma photons pass through the spatial-encoding sensor element of the additional coded sensor gamma imager module.

12. The method of claim 9, wherein reconstructing the SPECT image comprises reconstructing the SPECT image based at least in part on the first data and the second data.

13. The method of claim 9, further comprising calibrating the first detector with respect to the second detector before positioning the first detector module to receive first gamma photons from the subject.

14. The method of claim 13, wherein calibrating the first detector module with respect to the second detector module comprises:
positioning a positron emitting source between the first detector module and the second detector module at known distances and locations from the first detector module and the second detector module;
recording coincidence events of annihilation gamma photons emitted by the positron emitting source using the first detector module and the second detector module;
determining one or more calibrations based on the recorded coincidence events of annihilation gamma photons emitted by the positron source using the first detector module and the second detector module.

15. The method of claim 14, wherein the one or more calibrations include one or more of depth of interaction (DOI) calibration, crystal identification, energy calibration, timing correction, detector efficiency calibration, and system matrix calibration.

16. The method of claim 14, wherein:
reconstructing the SPECT image comprises reconstructing the SPECT image based at least in part on the first data and the one or more calibrations, and
reconstructing the PET image comprises reconstructing the PET image based at least in part on the first data and the second data and the one or more calibrations.

17. A method of calibrating a first gamma photon detector module with respect to a second gamma photon detector module comprising:
positioning the first gamma photon detector module opposite the second gamma photon detector module, the first gamma photon detector module including at least a light photon sensor coupled to at least an end of the first gamma photon detector module;
positioning a positron emitting source between the first gamma photon detector module and the second gamma photon detector module at a known first distance from the first gamma photon detector module and a known second distance from the second gamma photon detector module;
recording coincidence events of annihilation gamma photons from the positron source interacting with the first gamma photon detector module, and the second gamma photon detector module; and
determining timing factors based on the recorded coincidence events, the timing factors include one or more timing offsets for timing correction of the first gamma photon detector module, and one or more weighting factors for deriving trigger times of the coincidence events.

18. The method of claim 17, wherein the first gamma photon detector module includes a first light photon sensor at a first end of the first gamma photon detector module and a second light photon sensor at a second end of the first gamma photon detector module, the second end being further from the second gamma photon detector module than the first end;
recording coincidence events of annihilation gamma photons from the positron source interacting with the first gamma photon detector module, and the second gamma photon detector module comprises recording triple coincidence events of gamma photons from the positron source interacting with the first light photon sensor at the first end of the first gamma photon detector module, the second light photon sensor at the second end of the first gamma photon detector module, and the second gamma photon detector module; and
determining timing factors based on the recorded coincidence events comprises determining timing factors based on the recorded triple coincidence events.

19. The method of claim 17, wherein recording coincidence events of annihilation gamma photons from the positron source interacting with the first gamma photon detector module, and the second gamma photon detector module comprises recording double coincidence events of gamma photons from the positron source interacting with two different light photon sensor elements of the first gamma detector module, and the method further comprises:
determining depth of interaction (DOI) calibrations based at least in part on the recorded double coincidence events.

20. The method of claim 19, wherein determining DOI calibrations based at least in part on the recorded double coincidence events includes generating one or more histograms based on a ratio of energy levels of the two different light photon sensor elements for recorded double coincidence events.
